(19) United States
(12) Patent Application Publication
(10) Pub. No.: US 2026/0363579 A1
(43) Pub. Date: Nov. 19, 2026

(54) METHODS, SENSORS, AND SYSTEMS FOR MEASURING VENTILATION THRESHOLDS FOR IMPROVED FITNESS

(71) Applicant: Tyme Wear, Inc., Somerville, MA (US)

(72) Inventors: Arnar Freyr Larusson, Somerville, MA (US); Juan Carlos Morales Torrico, Somerville, MA (US); Arnar S. Hansson, Boston, MA (US); Gregory Laredo, Thornhill (CA)

(73) Assignee: Tyme Wear Inc., Somerville, MA (US)

(21) Appl. No.: 19/548,924

(22) Filed: Feb. 24, 2026

Related U.S. Application Data
(60) Provisional application No. 63/763,218, filed on Feb. 25, 2025.

(51) Int. Cl. A61B 5/113; A61B 5/00; A61B 5/0205; A61B 5/024; A63B 24/00

(52) U.S. Cl. CPC A61B 5/1135; A61B 5/0205; A61B 5/02438; A61B 5/6823; A61B 5/6831; A61B 5/725; A61B 5/7267; A63B 24/0062; A63B 24/0075; A61B 2562/0219

(57) ABSTRACT

Measurement of ventilation thresholds is used to assess cardiometabolic fitness and provide training guidance to users. The ventilation thresholds are measured using a wearable sensor that determines breathing rate, tidal volume, and minute ventilation. The measurement sensor may include a flexible sensor having a fixed stretchable length. The guidance provided to the user may be directed toward improving one or more ventilation thresholds.

### CROSS-REFERENCE TO RELATED APPLICATIONS

The present application claims the benefit of U.S. Provisional Patent Application Ser. No. 63/763,218, filed Feb. 25, 2025 under Attorney Docket No. T0961.70000US00 and entitled “METHODS, SENSORS, AND SYSTEMS FOR MEASURING VENTILATION THRESHOLDS FOR IMPROVED FITNESS,” which is hereby incorporated by reference herein in its entirety.

### BACKGROUND

### Field

The present technology relates to methods, sensors, and systems for measuring ventilation thresholds and using such measurements for improving fitness.

### Related Art

VO2max and ventilatory thresholds are physiological parameters that represent the maximum amount of oxygen and the fractional utilization of oxygen that an individual can achieve during exercise. VO2max and ventilatory thresholds can be measured using sophisticated lab equipment having flow sensors that sense the flow of oxygen and carbon dioxide. Such equipment is expensive and of limited accessibility, making it of limited use to those seeking to assess and improve upon their current fitness level.

If VO2max is the only physiological parameter sought, then sometimes it is indirectly determined using wearable sensors that measure heart rate, GPS, or accelerometry. An indirect determination of VO2max can be derived from heart rate by making various assumptions about the user and then applying known calculations linking the heart rate to VO2max. However, such calculations deriving VO2max from heart rate may not provide an accurate assessment of VO2max, and also do not provide any measure of ventilatory thresholds.

### BRIEF SUMMARY

Aspects of the present technology provide methods for accurately and consistently measuring ventilation thresholds of a user. In some aspects, a wearable sensor may sense breathing rate and tidal volume, allowing calculation of minute ventilation. Ventilation (ventilatory) thresholds may be measured by assessing inflection points in minute ventilation and/or breathing rate and/or tidal volume signals, thus providing an assessment of the user's fitness. Recommendations for activity type (including intensity) and duration may be determined based on the measured ventilation thresholds and provided to the user.

Aspects of the present technology provide stretchable wearable sensors for sensing the user's breathing rate and tidal volume. The stretchable wearable sensors are capacitive sensors in some embodiments and can be positioned on the user's chest to sense chest excursion. The capacitive sensors have a fixed initial length and may be coupled to a stretchable strap worn by the user. The stretchable portion of the stretchable strap that the sensor is coupled to may also have a fixed length. The capacitance of the stretchable sensor may vary as the sensor is stretched due to the user's breathing. The varying signal may be used to determine the user's breathing rate, and the amplitude of the signal may be used to determine tidal volume.

Aspects of the present technology provide systems for measuring a user's ventilation thresholds. The system may include a wearable sensor having a stretchable capacitive sensor, and a processing device such as a smartphone, tablet computer, cloud server, on-sensor microprocessor, or laptop computer. The wearable sensor may sense the user's breathing rate and tidal volume and calculate the minute ventilation. The wearable sensor may stream the breathing rate, tidal volume, and minute ventilation to another device such as the processing device or may process the breathing rate, tidal volume, and minute ventilation on a processing device of the sensor, either of which may measure the user's ventilation thresholds from the minute ventilation data. In some embodiments, a zone detection model may reside on the sensor, or alternatively on the processing device, that can analyze any combination of ventilation and heart rate signals to output an indication of the zone a user is in and/or make a determination of the values of the thresholds at the zone transitions. The processing device may present the user with information about his/her ventilation, such as a visual indication of the ventilation thresholds. The processing device may further present the user with recommendations for activity to improve the user's fitness based on improving one or more of the ventilatory thresholds or VO2 max (VO2max), or any combination of those physiological parameters.

According to some aspects, a wearable device may include: a strap; and a capacitive sensor coupled to the strap. The capacitive sensor comprises: a first end coupled to the strap; a second end coupled to the strap; and a stretchable portion between the first end and the second end, wherein the capacitive sensor has a capacitance that changes based at least in part on a length of the stretchable portion.

In some embodiments, the wearable device may be configured to measure chest wall excursion based at least in part on a change in the capacitance of the capacitive sensor. In some embodiments, the strap may include a stretchable segment. In some embodiments, the first end of the capacitive sensor may be coupled to a module having a housing. In some embodiments, the wearable device may further include an inertial measurement unit and/or an accelerometer in the housing of the module. In some embodiments, the wearable device may further include a heart rate sensor coupled to the strap. In some embodiments, the capacitive sensor may be facing an external surface of the strap. In some embodiments, the capacitive sensor includes silicone, urethane, and/or rubber. In some embodiments, the strap may be configured to surround an abdomen or thorax of a user. In some embodiments, the stretchable portion of the capacitive sensor may have a rectangular shape. In some embodiments, an amount of stretchable material for the stretchable segment of the strap is a fixed amount.

According to some aspects, a method is provided for measuring chest wall excursion using a wearable device comprising a capacitive sensor coupled to a strap. The method may include: obtaining a ventilation signal using the wearable device, wherein the ventilation signal is based at least in part on one or more changes in capacitance of the capacitive sensor; using one or more points of the ventilation signal to measure the chest wall excursion; and obtaining tidal volume based on the chest wall excursion.

In some embodiments, the one or more points of the ventilation signal may be inflection points. In some embodiments, the capacitive sensor may include a stretchable portion correlated to the tidal volume. In some embodiments, the one or more changes in capacitance of the capacitive sensor are based on the stretchable portion of the capacitive sensor being stretched by 20%-40% or 5%-60%. In some embodiments, the method may further include measuring one or more ventilatory thresholds based in part on minute ventilation and/or based in part on the tidal volume and/or breathing rate. In some embodiments, the one or more ventilatory thresholds include VT1 and VT2. In some embodiments, an additional ventilatory threshold, such as an endurance threshold or balance point, may be measured. In some embodiments, the ventilation signal is obtained while the strap is positioned on a user to surround an abdomen or thorax of the user. In some embodiments, the method may further include obtaining one or more measurements from an inertial measurement unit, an accelerometer, and/or a heart rate sensor coupled to the strap. In some embodiments, the method may further include filtering the ventilation signal to obtain the one or more points. In some embodiments, the method may further include using a trained model to extract one or more thresholds from a minute ventilation signal.

According to some aspects, a method of assessing fitness of a user and providing an individualized exercise recommendation to the user, may include: receiving from a wearable sensor worn by the user, minute ventilation data determined from a chest excursion signal sensed by the wearable sensor; measuring a first ventilation threshold (VT1), second ventilation threshold (VT2), and VO2 max for the user based on the minute ventilation data; comparing VT1, balance point (BP) or Endurance, VT2, and VO2 max to respective reference values; and based on the comparison, identifying at least one of VT1, BP or Endurance, VT2, or VO2 max as a target for the exercise recommendation; and providing the user the individualized exercise recommendation having a target exercise intensity and duration for multiple physiological zones associated with VT1, BP or Endurance, VT2, and VO2 max.

In some embodiments, measuring VT1, VT2, BP or Endurance, and VO2 max comprises using a neural network to perform the measurement. In some embodiments, measuring VT1, VT2, BP or Endurance, and VO2 max comprises using fixed benchmarks that have been predetermined for that user, such as a heart rate or ventilation value associated with the user's threshold that was determined in a prior threshold test or one or more workouts. In some embodiments, the wearable sensor comprises a stretchable sensor, and the chest excursion signal is a varying signal representing stretching of the stretchable sensor as the user breathes. In some embodiments, identifying at least one of VT1, BP or Endurance, or VT2 as a target for the exercise recommendation involves identifying VT1 and not BP or VT2 as the target for the exercise recommendation. In some embodiments, identifying at least one of VT1 or VT2 as a target for the exercise recommendation involves identifying VT2 and not VT1 as the target for the exercise recommendation. In some embodiments, identifying at least one of VT1 or VT2 as a target for the exercise recommendation involves identifying both VT1 and VT2 as the targets for the exercise recommendation. In some embodiments, providing the user the individualized exercise recommendation comprises providing a visual recommendation on a graphical user interface of the user's smartphone, smartwatch, GPS-tracking watch, and/or bike computer.

According to some aspects, a computer readable storage medium is provided that comprises computer readable instructions configured to be executed by a processor to cause the processor to perform any of the methods of the preceding aspects.

### BRIEF DESCRIPTION OF DRAWINGS

Various aspects and embodiments of the technology will be described with reference to the following figures. It should be appreciated that the figures are not necessarily drawn to scale. Items appearing in multiple figures are indicated by the same reference number in all the figures in which they appear.

FIG. 1 is a flowchart illustrating a method of measuring ventilation thresholds using data received from a wearable sensor, according to some embodiments.

FIG. 2 is a graph illustrating measured ventilation thresholds and associated intensity zones.

FIG. 3 is a system including a wearable sensor configured to measure chest wall excursion in communication with a processing device, according to some embodiments.

FIG. 4 is a block diagram of a system comprising a wearable sensor, a processing device, and optionally a server, for measuring ventilation thresholds and providing exercise recommendations, according to some embodiments.

FIG. 5 is a graphical user interface (GUI) of a processing device illustrating presentation of ventilation threshold data, according to some embodiments.

FIG. 6 is an example recommendation provided on a GUI to a user for a training program, according to some embodiments.

FIG. 7 shows an example training recommendation provided on a GUI to a user for a training program, according to some embodiments.

FIG. 8 shows an example recommendation provided on a GUI to a user for a training program with improvements in each category relative to FIG. 6, according to some embodiments.

FIG. 9 shows an example recommendation provided on a GUI to a user for a training program with improvements in some categories relative to FIG. 8, according to some embodiments.

FIG. 10 shows a dashboard provided on a GUI to a user including a fitness profile, individualized training distribution, and personalized workout recommendation, according to some embodiments.

FIGS. 11A-11C show positioning of a strap having one or more wearable sensors on a user, according to some embodiments.

FIG. 12 shows a stretchable and non-stretchable portion of the wearable sensor 1240 of FIGS. 11A-11C, according to some embodiments.

FIG. 13 shows an example of fit adjustment portions, according to some embodiments.

FIG. 14 shows an example coupling of a stretchable and non-stretchable portion, according to some embodiments.

FIG. 15 shows a direction of stretchability for the stretchable portion of the strap shown in FIGS. 11A-11C, according to some embodiments.

FIG. 16 shows a force-elongation curve for a variety of materials.

FIG. 17 illustrates a diagram representing the capacitive sensor in a relaxed and stretched state.

FIG. 18 shows a cross-sectional view of layers of the capacitive sensor, according to some embodiments.

FIG. 19 shows different portions of the stretchable portion of the strap, according to some embodiments.

FIG. 20 shows an exploded view of the respective sensors of the stretchable and non-stretchable portions of FIGS. 11A-11C, according to some embodiments.

FIG. 21 shows inner sides of a module and connector coupled to the capacitive sensor, according to some embodiments.

FIG. 22 illustrates a computing system configured to perform aspects of the present technology.

FIGS. 23-28 show additional example displays provided on a GUI to a user, according to some embodiments.

FIG. 29 shows an example system including a wearable sensor with a single housing, according to some embodiments.

FIG. 30 is a flowchart illustrating a method of measuring ventilatory thresholds using data collected with a wearable sensor, according to some embodiments.

### DETAILED DESCRIPTION

Aspects of the present technology provide methods, sensors, and systems for measuring ventilation thresholds of a user and using such measurements for improving the user's fitness.

The inventors recognized that heart rate does not correlate well with ventilatory (ventilation) thresholds that represent an individual's fractional utilization of their VO2 max. For example, while a user's heart rate may increase during exercise, the volume inhaled or exhaled by the user may drop. Moreover, two people can have the same VO2 max but different fractional utilization of their VO2 max. Thus, in many instances using heart rate does not provide an accurate assessment of VO2 max, and furthermore does not provide an accurate assessment of exertion level or fitness level.

According to some methods described herein, the fractional utilization of a person's aerobic capacity can be assessed, which may be done by determining ventilatory thresholds. In some embodiments, two or more ventilatory thresholds may be determined from a breathing signal, such as the minute ventilation signal. The aerobic capacity may be represented as VO2 max as described herein. Ventilatory thresholds can determine fractional utilization of aerobic capacity such that breathing uncovers the ventilatory thresholds. Measuring breathing includes both measuring breathing rate and tidal volume which produces minute ventilation. Minute ventilation responds to changes in muscle substrate utilization. Muscle substrate utilization is determined by the ratio of fat versus carbohydrates (“carbs”) that the muscle is using, wherein fat is efficient and carbs are inefficient. The ratio of fat to carbs determines the efficiency of the work performed. By targeting these transitions of decreasing efficiency in training, the muscles of the individual can adapt by becoming more efficient, thereby increasing fitness level, including improving their ability to go faster for longer. The inventors have recognized that the ventilatory thresholds are the measurable phenomena that exposes these transitions such that they can be measured and targeted in training.

The inventors recognized that the minute ventilation exhibited by an individual across increasing exercise intensity exhibits three inflection points primarily driven by the increasing CO2 production as a result of increasing carbohydrate utilization within the working muscles. One inflection point may be when maximum fat utilization is reached and carbohydrate utilization begins to increase in order to continue to match energy demands of the increasing exercise intensity. Another inflection point may be the aerobic threshold occurring when fat utilization begins to decline causing carbohydrate utilization to increase even more. Another inflection point may be the anaerobic threshold where fat is minimal and carbohydrates make up the entire production which eventually becomes unsustainable. These three inflection points occur in that order as a user increases exercise intensity. That is, as a user increases exercise intensity, the user's minute ventilation will first experience the threshold prompted by maximal fat utilization being reached and carbohydrate utilization beginning to increase, then the aerobic threshold prompted by fat utilization dropping off with a resulting increase in carbohydrate consumption, and then the anaerobic threshold prompted by carbohydrates making up the majority of production which eventually becomes unsustainable.

For purposes of this document, the three thresholds exhibited in the user's ventilation corresponding to increased intensity are referred to as the “low ventilatory threshold,” (or simply the “low threshold”), the “medium ventilatory threshold,” (or simply the “medium threshold”) and the “high ventilatory threshold,” (or simply the “high threshold”) since they appear in that order in the user's ventilation as a function of exercise intensity. That is, the “low ventilatory threshold” (or simply “low threshold”) is the first threshold that occurs at the lowest exercise intensity. The “medium ventilatory threshold” (or simply “medium threshold”) is the next threshold that occurs at increased exercise intensity. The “high ventilatory threshold” (or simply “high threshold”) is the ventilatory threshold that appears at even higher exercise intensity and prior to reaching VO2 max. In some instances, the low ventilatory threshold may result from a physiological change experienced by the user in which maximum fat utilization is reached and carbohydrate utilization begins to increase in order to continue to match energy demands of the increasing exercise intensity. The medium threshold may result from a physiological change at which maximum fat utilization occurs at the aerobic threshold. Between the low and the medium thresholds is sometimes referred to as the “fat plateau.” The high ventilatory threshold may result from a physiological change where lactate production becomes unsustainable at the anaerobic threshold. The user's ventilation as described herein can be tracked with a device which produces a minute ventilation signal. The slope of the minute ventilation signal leading into successive ventilation thresholds (low, medium, and high) will increase for each individual such that for any given individual the slope leading into the medium ventilatory threshold will be higher than the slope leading into the low ventilatory threshold, and the slope leading into the high ventilatory threshold will be higher than the slope leading into the medium ventilatory threshold. Two or more of the low, medium, and high ventilatory thresholds may be used as described herein to analyze the fractional utilization of a person's aerobic capacity.

The low, medium, and high ventilatory thresholds as described above are sometimes called by different names in the field. For example, “Endurance” may be used to refer to the low ventilatory threshold. “VT1” may, at times, be used in the field to refer to the low ventilatory threshold and, at other times, to refer to the medium ventilatory threshold. “VT1” is sometimes also referred to as “the aerobic threshold” or “sweet spot.” “VT2” may, at times, be used in the field to refer to the medium ventilatory threshold and, at other times, to the third ventilatory threshold. “VT2” is sometimes also referred to as the “anaerobic threshold,” “critical power,” or “FTP.”

The methods described herein involve measuring the user's ventilation (ventilatory) thresholds using a combination of Tidal volume, breathing rate, and minute ventilation data from a wearable sensor. According to the techniques described herein, two or more inflection points in a breathing signal may be determined. The inflection points represent physiological changes as described above.

As described herein, the minute ventilation data can be accurately and consistently determined using a stretchable capacitive sensor fixed to a strap having a fixed initial stretchable length. The user may wear the strap around their chest or, more generally, torso, and the user's breathing may be sensed by the user's chest excursion. The chest excursion signal provides breathing rate and, using sensors of the types described herein, an accurate indication of tidal volume. The minute ventilation can be calculated from the breathing rate and tidal volume, and the user's ventilation thresholds (low, medium, and high as described above, or referred to by names such as VT1, BP or Endurance, and VT2) and VO2 max may be measured manually or using an automated tool such as a neural network trained to identify such inflection points in the ventilation signal. Moreover, measuring one or more ventilatory thresholds may be based on the tidal volume and may comprise measuring the one or more ventilatory thresholds based on a minute ventilation signal. Two of the ventilation thresholds (low, medium, and high as described above, or referred to by names such as VT1, BP, Endurance, or VT2) and/or VO2 max may be used and/or measured in some examples. In some embodiments, the minute ventilation may be calculated using breathing rate times tidal volume. In some embodiments, linear regression, two-line best fit, three-line best fit, four-line best fit, or a Gaussian model that assess break point (looking for 1, 2, or 3 break points) may be used to extract one or more thresholds of a minute ventilation signal.

According to some aspects, the present technology provides methods that may include segmenting an individual's ventilation response to exercise into 4 distinct segments. Each segment may be defined by the absolute value of ventilation and the rate of increase within each segment. The transitions between these segments may be referred to as low, medium, and high thresholds as described above, or referred to by names such as VT1, BP or Endurance, VT2, and VO2 max, corresponding to ventilatory thresholds and VO2 max.

The direct measurement of the user's ventilation thresholds (low, medium, and high as described above, or referred to by names such as VT1, BP or Endurance, and VT2), allows for user-specific assessments of fitness to be made, which in turn allows for tailored activity recommendations. In contrast to calculating the ventilation thresholds, for example based solely on population statistics, aspects of the present technology provide increased personalization with improved activity recommendations. Moreover, aspects of the present application provide versatile wearable sensors which may be worn by users in day-to-day activity and exercising situations, thus facilitating regular and low-cost measurement of fitness. Aspects of the present application allow for continuous monitoring of fitness and measurement of ventilation thresholds.

The inventors have recognized that improving a person's fitness level is best done with individualized training. A person's fitness may depend on individualized physiological parameters, such as ventilation thresholds. The values of such thresholds, and their relative values, for a given individual may differ from statistical values which may be based on population averages. Thus, to provide individualized training, it is desirable to know the set of appropriate intensities and the duration those intensities should be maintained to create an adaptation in the person's physiology to improve their fitness level. This, in turn, means assessing where these physiological boundaries are for each individual, how to best target them in training, comparing them against each other to determine where there is room to improve, and how to best target these boundaries/thresholds in real-time to induce the best possible adaptation. Training at an identified intensity may not elicit improvement because the person's fitness at that intensity may not have the room to improve and its further improvement is being blocked by another aspect of the person's physiology which must be improved first to create the ability for further improvement.

The inventors have further recognized that determining how an individual can improve their fitness is furthered by accurately determining ventilatory threshold(s), which determination relies on an accurate and reliable ventilation signal. Historically, getting an accurate and reliable threshold assessment has been challenging because it either involves lab testing to acquire an accurate ventilation signal combined with manual expert analysis of the data collected or the use of invasive test equipment in the field with blood samples analyzed by a lactate meter to identify appropriate training targets. Non-measurement approaches use formulas that make statistical assumptions applied to a person's activity data or use trend analysis and comparisons of datasets to make predictions or assessments of someone's fitness level. The lab methods and invasive methods are cumbersome, costly and time consuming. The estimates made by formulas with or without physiological data are prone to high error margins and inconsistent assessments, making them ineffective at assessing fitness level and changes therein or in prescribing training.

### Determining Ventilatory Thresholds

The most common way to identify the intensities described herein is by assessing inflection points in a person's respiration or lactate that indicate a change in how efficiently muscles are consuming their fuel resources. As described further herein, by detecting at least two (2) or three (3) inflection points in a ventilation signal, the area between the inflection points can be designated as “training zones” and the inflection point detections themselves can be used as “intensity targets.”

Both respiration and lactate increase due to increased carbohydrate consumption. Carb consumption results in higher outputs of CO₂ causing ventilation to increase to maintain PH balance in the body. Increased carb consumption also leads to higher output of lactate which is created by CHO→Pyruvate+H+→Lactate mechanism. The workload at these ventilation and/or lactate inflection points can be used to target them during training. The appropriate workload can be prescribed via the ventilation parameters, heart rate, power output, speed, lactate value, or a host of other metrics associated with that specific workload at a given ventilatory or lactate inflection point. Not all metrics are equally good for prescription, for example it has been shown that ventilation is a more appropriate workload for targeting the first ventilatory threshold compared to heart rate or power or speed. The reason for this is that heart rate can be affected by stressors like dehydration, psychological stress, lack of sleep, and a host of other factors that can cause the heart rate value at a given ventilation or lactate threshold to shift day-to-day. Power is also less reliable because muscular fatigue can affect a person's ability to output a given power over the course of a workout, meaning that the power output shifts downward at a given metabolic threshold over the course of a workout. Ventilation on the other hand is less affected by external stressors and holds “truer” to the metabolic output since the volume that is being exchanged represents the net sum of the substances, including CO₂ and O₂, that the body is exchanging to perform the work at hand.

How Metrics Associated with Ventilation Determine Fitness Progress/Regression

Improvement or regression in fitness level can then be assessed by observing any changes in the inflection points described above, and their associated metrics, over time. An inflection point occurring at a higher workload indicates improved fitness, while the opposite indicates a regression in fitness level.

When assessing the breakpoints with ventilation, the inventors recognized that it is beneficial to know breathing rate and tidal volume accurately. The product of breathing rate and tidal volume is minute ventilation, or the volume over time breathed in and out. It is beneficial to have both because when CO₂ production increases in any given individual, how their breathing system decides to release it is either by increasing their breathing rate, increasing their tidal volume, or a unique combination of the two. Thus, the same change in Minute Ventilation can be accomplished by increasing tidal volume while keeping breathing rate the same or even decreasing breathing rate, and vice versa, or through any number of combinations of the two increasing/decreasing to achieve a given minute ventilation. Therefore, if only one of the signals is assessed, then the true inflection point can be missed in situations in which the inflection point is driven by the signal that is not being measured. With only one or the other of breathing rate and tidal volume, an assessment of these inflection points cannot be accurately or reliably made for all individuals.

### Consistent Relative Tidal Volume

Determination of the inflection points described above may be facilitated by ensuring a minimum level of accuracy of relative tidal volume. To reliably assess the ventilation inflection points, it is unnecessary to have a precise absolute value of the metrics being measured, e.g., breathing rate and tidal volume, but an accurate measurement of the relative change during the measurement period in both breathing rate and tidal volume is desirable. If, for example, the tidal volume changes 15% over a given period of time during a measurement it is not necessary for the exact liters exchanged to be accurately measured, however, it is beneficial that the relative change of 15% is reliably measured within a reasonable margin of error. If not, then any error or discrepancy between measured and actual volume exchanged can obscure a threshold detection.

The degrees of accuracy and reliability for relative tidal volume can be expressed in terms of:
Level 1: relative change from one breath to the next
Level 2: relative change across multiple breaths
Level 3: relative change across all breaths in a given measurement period
Level 4: relative change across multiple measurement periods, but not all
Level 5: relative change across all measurement periods-effectively an accurate absolute measurement

### Consistent Absolute Tidal Volume

Identifying the absolute ventilation value at a given threshold inflection point facilitates using the ventilation value to prescribe and guide workouts in real-time. For example, knowing the absolute ventilation value facilitates prescribing to a user that they should, for example, stay between X and Y ventilation output for Z duration, where X, Y, and Z are variables. Moreover, the method used to measure the value of X and Y ventilation should stay consistent from one day to the next to ensure reliability and effectiveness of the prescription of using a ventilation parameter to guide workouts. The application of guiding a workout based on ventilation is not possible if only the relative change during a given measurement period is known.

### Fixed Stretchable Portion

According to aspects of the present technology, a consistent tidal volume is ensured by ensuring that the sensing element is fixed to a stretchable element with a fixed initial length and a homogenous or known stretch modulus. If either the initial length or the stretch modulus is variable in the operating range where the data is being acquired, then the values from the sensor cannot be relied upon to assess either accurate relative tidal volume change or absolute volume change.

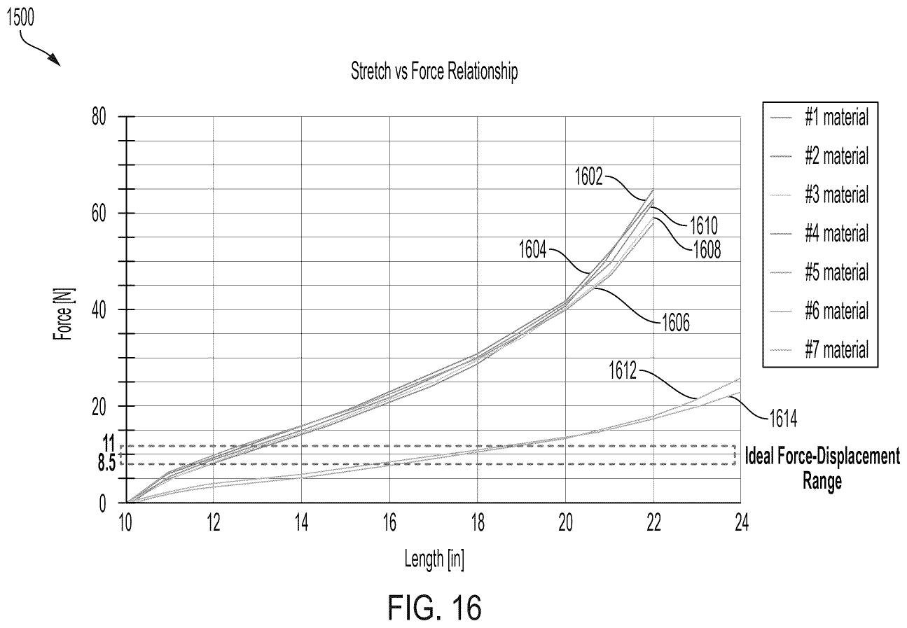

An ideal strap material may have a constant stretch modulus such as a consistent stretch modulus over time and over all stretch cycles. However, a stretch modulus may vary for most elastic materials. Characterizing the stress-stretch relationship (such as described in relation to FIG. 16) across the operating range of the system allows for any changes over time to be accounted for, such as using a linearized model. Further, the modulus of most elastic bands will change over time as stretching introduces a shift in the stress/strain relationship, such as a fabric strap getting softer over N stretch cycles, such that less stress is required for the same elongation. Most strap materials will have a settling effect where after N cycles, the changing of the stretch modulus will reach a limit. In some embodiments, materials can be pre-conditioned to ensure that the change of the stretch modulus has reached the limit before it is used by a user. There can be an acceptable change in elastic modulus over time, including changes caused by fatigue that do not alter the resulting stress-stretch relationship so much that it causes a significant error in the sensor reading. There is, however, unacceptable change in the elastic modulus that will introduce an error in the resulting sensor reading that is significant enough to produce a reading inconsistent with previous readings. The inventors recognized that the elastic modulus change over time within the operating range of the elongation may be accounted for in the measurements and is ideally minimized.

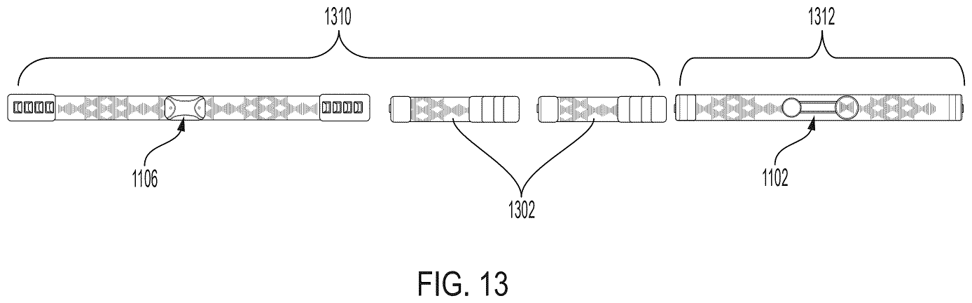

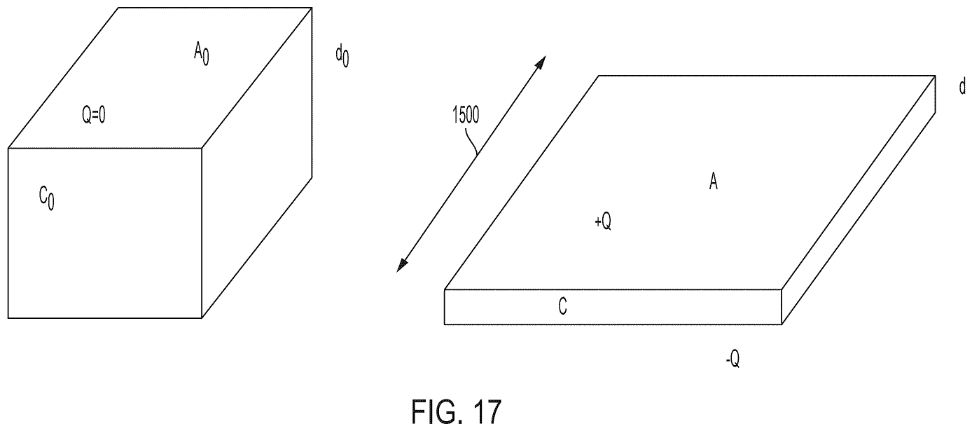

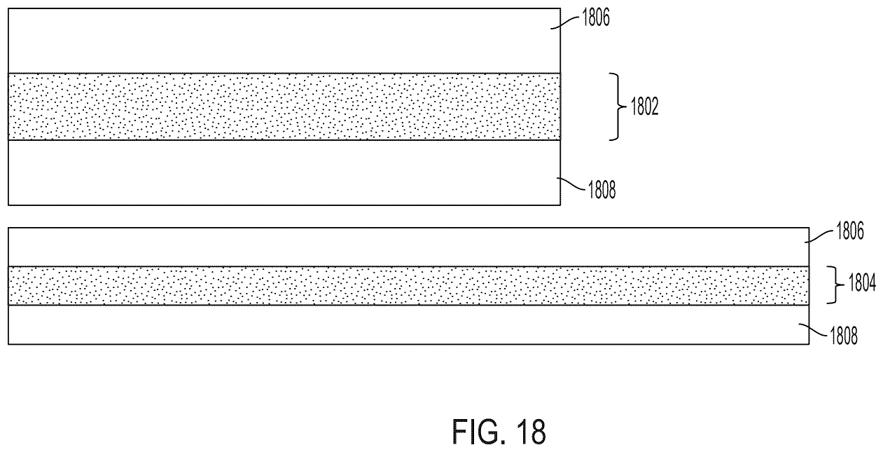

The inventors have developed a stretchable sensing element that has a known initial length that measures the expansion and contraction of the chest cavity of a person and an algorithm that takes in the signal and analyzes it for peaks and valleys, extracting the ventilation rate from the peak-to-peak time and the volume based on the amplitude between valley and peak. The sensor measures the capacitance change resulting from the stretchable element expanding and contracting with the chest cavity. As the chest walls expand and contract, the sensing element comes closer together and then goes farther apart, respectively, causing a capacitance change that corresponds to the expansion and contraction of the chest. The surface area of the sensor may change due to this stretching which causes a corresponding capacitance change, as described herein in relation to FIGS. 17-18. The wearable device may be configured to measure chest wall excursion based at least in part on a change in the capacitance of the capacitive sensor. The relationship between change in surface area and capacitance change is highly linear and consistent. The sensor can be of any length, provided it stretches as the chest expands and contracts. If the sensor is attached to other elements to fasten it around the chest, then these elements are provided as non-stretchable segments as described further in relation to FIG. 13. In some embodiments, the sensor is a 50 mm long sensor with electronics on one end that receive, store, process, and transmit the signal. The sensor can be attached anywhere along the vertical axis of the user's rib cage. For example, the sensor may be attached to a vest, shirt, or strap. The sensor may be permanently attached to the vest, shirt, or strap, and it can also be detachable via hook and loop (e.g., Velcro), snaps, or temporary adhesive. The sensor, according to some embodiments, is attached between two anchor points on either side of the sensor and when the chest wall expands the two anchor points move further apart, and when they contract, the two anchor points come back together.

According to an aspect of the present technology, an algorithm may be trained to recognize the signature in the ventilation data that corresponds to the transition of certain metabolic states: State 1: where the ventilation signal is relatively stable; State 2: where the ventilation signal is greater than in State 1 and less stable; State 3: a third state where the ventilation signal is higher and increases at a faster rate than in the second state; and State 4: a fourth state where the ventilation is higher and increases faster than in the third state. If the workload were to be “clamped” within any of these states, the signal would stabilize in some cases for some amount of time, and then given enough time, it would begin to increase again. The duration of stability is directly correlated with the state, meaning the stability is maintained the longest in the first state and shortest in the fourth state. Thus, there are multiple features that describe a ventilation signal that may be important to assessing an individual's metabolic state: the rate of change when the external workload is increasing; the rate of change when the external workload is decreasing; the rate of increase when the external workload is held constant; and the absolute value of ventilation when external workload is increasing, decreasing, or is constant.

The following is relevant to these metabolic states: 1. The rate at which the signal increased or decreased to get to a certain level, and 2. The absolute value of the ventilation defines the state the person is in.

Points 1 and 2 above differ greatly between people, and within a person these can change as fitness level changes. Thus, in at least some situations it is important to benchmark 1 and 2 for a given individual, and to have a way to continue to update the benchmark over time.

Each of the transitions identified between the metabolic states is used to determine what effort level each person is at relative to their known metabolic states. Associated data like HR, Pace, Power, Oxygen saturation, lactate, etc. can be used as associated physiological markers.

The breathing rate, described herein, can map to the stress level of the individual. The relationship between breathing rate and stress can be determined independent of the O2 and CO2 demands of the task at hand. This is due in part to a user's body controlling the oxygen that comes in and CO₂ that goes out, where the breathing rate registering as a user's stress builds and the Tidal volume calibrating itself against the breathing rate in order to achieve the required minute ventilation needed to service the metabolic demand. Moreover, breathing describes the stress state (breathing rate) and the metabolic state (minute ventilation). The stress and/or the workload level of the individual can be factored into a recommendation to the individual. Further, a stress score, strain score, mobilization score, or load score based in part on the breathing data described herein can be determined and/or presented to a user.

Aspects of the present technology provide sensors, systems, and methods for making such assessments of metabolic activity and tailoring exercise recommendations accordingly.

The aspects and embodiments described above, as well as additional aspects and embodiments, are described further below. These aspects and/or embodiments may be used individually, all together, or in any combination of two or more, as the application is not limited in this respect.

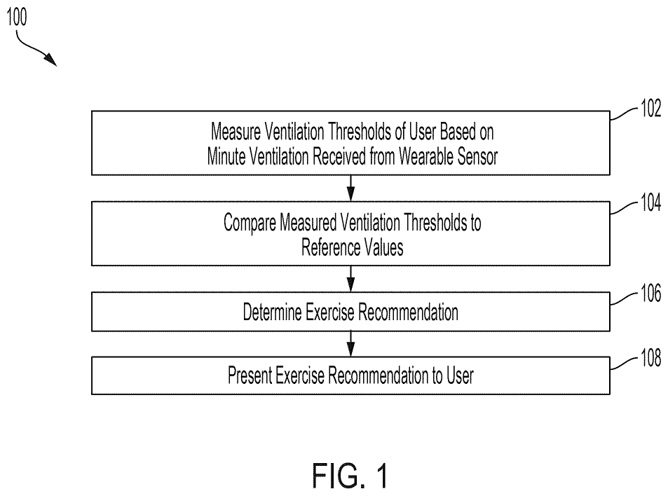

FIG. 1 is a flowchart illustrating a method of measuring ventilation thresholds using data received from a wearable sensor. The wearable sensor may be of the types described herein. The data received from the wearable sensor may be ventilation data, for example minute ventilation. The method may be performed using a processing device, such as a smartphone, laptop computer, tablet computer, or other processing device.

The method 100 may include an act 102 of measuring ventilation thresholds (low, medium, and high as described above, or referred to by names such as VT1, VT2, BP or Endurance) and/or VO2 max of a user based on minute ventilation received from the wearable sensor.

The method 100 may include an act 104 of comparing measured ventilation thresholds (low, medium, and high as described above, or referred to by names such as VT1, VT2, BP or Endurance) and VO2 max to reference values.

The method 100 may include an act 106 of determining one or more exercise recommendations. The determination may be based at least in part on the measurements of acts 102 and 104.

The method 100 may include an act 108 of presenting the one or more exercise recommendations (from act 106) to a user.

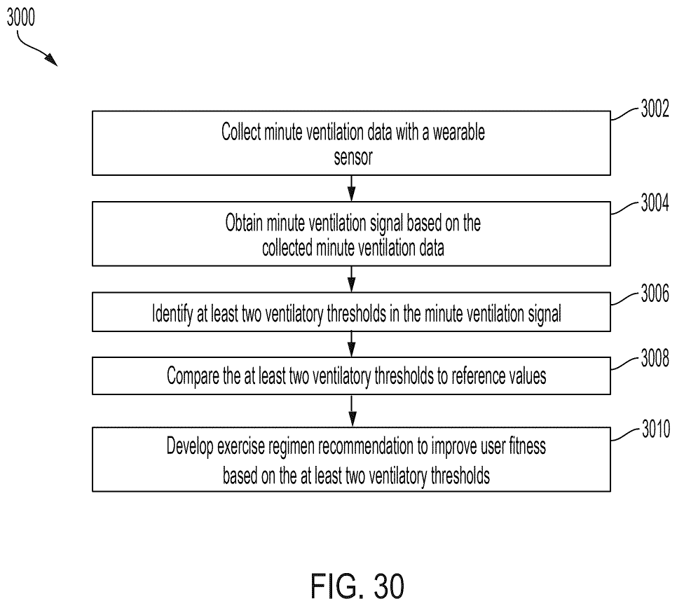

FIG. 30 is another flowchart illustrating a method 3000 of measuring ventilatory thresholds using data collected with a wearable sensor. The wearable sensor may be of the types described herein. The data received from the wearable sensor may be ventilation data, as described herein. The method may be performed using a processing device, such as a smartphone, laptop computer, tablet computer, or other processing device.

The method 3000 may include an act 3002 of collecting minute ventilation data with a wearable sensor. The minute ventilation data may be collected while the wearable sensor is worn by a user. Further, the minute ventilation data may be collected during an exercise session of the user.

The method 3000 may include an act 3004 of obtaining at least one minute ventilation signal based on the collected minute ventilation data. In some embodiments, act 3004 includes receiving from the wearable sensor worn by the user, minute ventilation data determined from a chest excursion signal sensed by the wearable sensor. The ventilation signal may be based at least in part on one or more changes in capacitance of the capacitive sensor. In some embodiments, one or more points of the ventilation signal may be used to measure chest wall excursion, as described further herein. The one or more points may be inflection points. The method may include filtering the ventilation signal to obtain the one or more points. In some embodiments, tidal volume may be obtained based on the chest wall excursion.

As shown in FIG. 30, the method 3000 may include an act 3006 of identifying at least two ventilatory thresholds in the minute ventilation signal. The at least two ventilatory thresholds may include low, medium, and high ventilatory thresholds. Using another naming convention, the at least two ventilatory thresholds may include first, second, and third ventilation thresholds such as VT1, VT2, and VO2 max of a user as well as BP or Endurance. One or more ventilatory thresholds may be measured based in part on the tidal volume. A first ventilation threshold (such as but not limited to VT1), second ventilation threshold (such as but not limited to VT2), and VO2 max for the user may be measured based on the minute ventilation data.

The method 3000 may include an act 3008 of comparing the at least two ventilatory thresholds to reference values. As a non-limiting example, the method may include comparing VT1, VT2, and VO2 max to respective reference values.

The method 3000 may include an act 3010 of developing an exercise regimen recommendation to improve user fitness based on the at least two ventilatory thresholds including the comparison results of act 3008. As a non-limiting example, based on the comparison, at least one of VT1, VT2, or VO2 max may be identified as a target for the exercise recommendation. Identifying the target may include identifying one of VT1 and VT2 but not the other or identifying both as the targets. In some embodiments, the method may include providing the user the individualized exercise recommendation having a target exercise intensity and duration for multiple physiological zones associated with the ventilatory thresholds (such as but not limited to VT1 and VT2) and/or VO2 max. In some embodiments, providing the user the individualized exercise recommendation includes providing a visual recommendation on a graphical user interface of the user's smartphone, as described elsewhere herein.

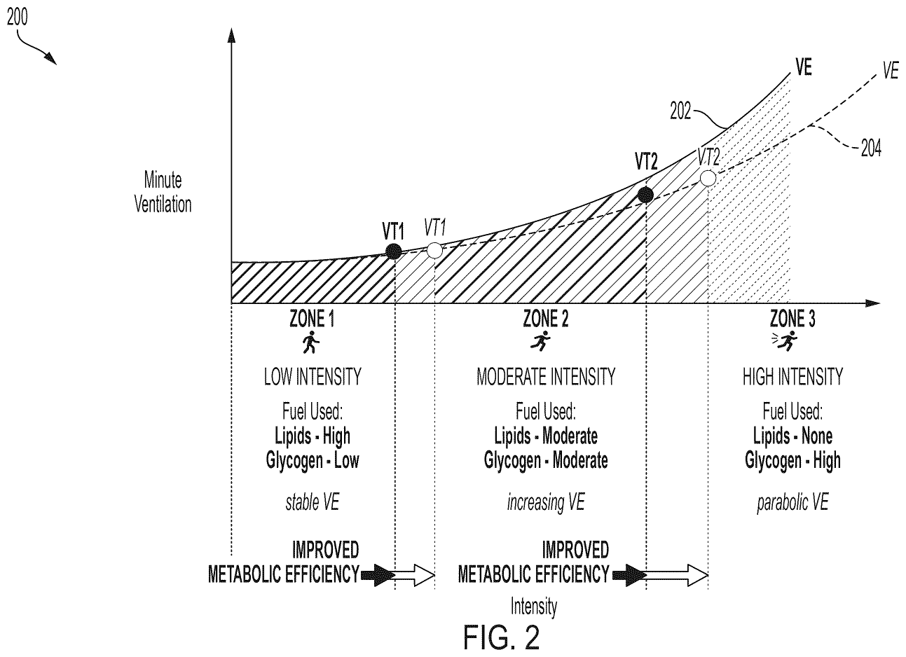

FIG. 2 is a graph 200 illustrating measured ventilation thresholds and associated intensity zones. The graph may plot intensity and minute ventilation. The zones may be based in part on known user information including age, gender, and past activities in addition to a current session for the user.

As shown in FIG. 2, metabolic efficiency can be improved as the ventilation thresholds (VT1 and VT2) increase in intensity in Zone 1 and Zone 2, for example. As discussed herein, the ventilation threshold naming convention may differ, and VT1 and VT2 in FIG. 2 may be referred to as medium and high ventilation thresholds, among other names. Minute ventilation (VE) 202 may be measured at a first time. Minute ventilation (VE) 204 may be measured at a second, later time after a period of training directed to improving metabolic efficiency, such as by using the techniques described herein.

Breathing during exercise is predominantly driven by CO₂ levels in the body. Breathing is driven by inputs including the metabolic demand of a task at hand which drives the need to inhale oxygen and exhale CO₂. The metabolic demand is primarily met via minute ventilation. Stress state of the individual will drive the breathing rate response such that more stress generally increases breathing rate. If stress increases while the metabolic load is held constant, then the tidal volume will decrease proportionally to avoid hyperventilation.

CO₂ is the waste byproduct of cellular respiration as an individual's muscles metabolize their fuel resources, predominantly lipid and glycogen, to meet the energy demand of the work being performed. In fact, when someone loses weight, it is in part because they have breathed out more carbon atoms in the form of CO₂ than the carbon atoms they have retained from the food they've consumed. Understanding how CO₂ drives minute ventilation is key to understanding how breathing gives us a window into individuals' metabolic efficiency.

As exercise intensity goes from low to high, the body traverses three phase shifts driven by its fuel consumption. When energy demand is relatively low, a person's muscles are able to rely in large part on lipids. Lipids produce less volume of CO₂ for each gram of lipid that is consumed as compared to the amount of CO₂ produced by each gram of carbohydrate. This results in lower levels of minute ventilation since the lungs only need to supply enough air to satisfy the O₂ demand. Lipids are both abundant and energy dense. The body stores approximately 2 week's worth of lipids at a given time and lipids produce 2× more energy from one gram compared to 1 gram of glycogen. However, it is relatively harder to extract energy from lipids, and as exercise intensity increases to a moderate level, the body begins to transition to a more readily available fuel source, glycogen. For each gram of glycogen consumed, 30% more CO₂ is produced, and only half the energy is generated compared to lipid consumption. The increased CO₂ production drives higher ventilation to get rid of the excess. This transition is referred to as low ventilation threshold, among other names including the First Ventilatory Threshold (VT1).

As should be appreciated, the ventilation thresholds, shown in FIG. 2 and described elsewhere herein, can have different names, but they are related to physiological changes as described herein. Therefore, while naming conventions may be assigned to ventilatory thresholds herein, the ventilatory thresholds may be named differently in other contexts. The same points, however, are identified independent of the naming conventions. As an example, a first shift during exercise may occur at a first inflection point. The first inflection point may be identified as a first threshold and may be referred to as “VT1” or “Endurance.”

Once the first threshold is traversed and intensity continues to rise, lipid consumption will eventually start to decline. The more metabolically efficient someone is (e.g., higher fitness level), the higher the intensity is at which the lipid consumption declines. The start of the lipid decline may, in some contexts, be referred to as medium ventilation threshold among other names including the Balance Point or, in other contexts, VT1. As lipid consumption declines, it is replaced by sufficient glycogen to match the energy producing capacity of the lipid decline, if workload is increasing, then the energy requirement from the increased workload is met by increased glycogen consumption. The increased glycogen consumption causes ventilation to begin increasing at a greater rate to remove excess CO₂ that is being produced. Each gram of lipid needs to be replaced by 2 grams of glycogen which in turn produces 60% more CO₂. This compounding effect increases CO₂ production and drives ventilation higher, eventually producing a parabolic increase.

As exercise intensity climbs higher, the body eventually transitions away from lipids entirely while increasing its use of glycogen to meet the energy demand of the workload. At this point, the metabolic system goes into a negative feedback loop where fuel consumption has reached unsustainable levels while relying on one of the body's most scarce fuel sources. This transition is called the high ventilation threshold among other names including Second Ventilatory Threshold (VT2).

Beyond this point, the depletion of glycogen stores and/or the acidification from compounding CO₂ levels, and/or build up of H+ ions in the muscles, along with a host of other associated effects of unstable metabolic activity, will eventually contribute to the reduction of the muscle's ability to perform work, forcing the body to slow down. At this point, improvement in a user's efficiency may be achieved given that certain loads are on the user.

In summary, with each transition, glycogen consumption increases and drives CO₂ production and the corresponding increase in minute ventilation to expel it. By measuring breathing, specifically minute ventilation, these transitions can be assessed and targeted in training. The body's ability to delay these transitions is a result of greater ability to consume lipids, and more efficient utilization of glycogen which is referred to as the Glycogen Sparing effect. This phenomenon is achieved by calibrating training intensities and durations around each person's unique transition points. It is the underlying mechanism by which fitness is increased in order to go faster for longer.

Breathing provides an accessible method to assess metabolic output and efficiency. Aspects of the present technology provide for measuring the increase in Minute Ventilation to identify each of the transitions between the four phases, and provides actionable and individualized targets to improve metabolic efficiency. Changes over time can be tracked by how the ventilation response changes.

Low, medium, and high ventilation thresholds, or referred to by names such as VT1, BP, Endurance, or VT2, and/or VO2 max may be visually identified from the breathing signals obtained during a ramp test protocol or obtained using a neural network algorithm that has been trained to identify the inflection points based on labeled datasets (e.g., tens of labeled datasets or hundreds of labeled datasets) where the thresholds have been visually identified by an experienced practitioner. In some embodiments, a model has been trained using data and an algorithm other than a neural network. In some embodiments, the model, including a neural network or other type of model, identifies how a user's breathing pattern changes over time. The model may analyze a signal on a breath-by-breath basis. The model may analyze heart rate (such as in beats per minute) and minute ventilation, as described herein. The trained model may be used to extract one or more thresholds from a minute ventilation signal.

In some embodiments, measuring ventilation thresholds such as low, medium, and high as described above, or referred to by names such as VT1, VT2, BP or Endurance, and VO2 max comprises using fixed benchmarks that have been predetermined for that user, such as a heart rate or ventilation value associated with the user's threshold that was determined in a prior threshold test or one or more workouts. In some embodiments, the fixed benchmarks may be determined using a calibrated data set without a prior threshold test or in combination with data from a prior threshold test.

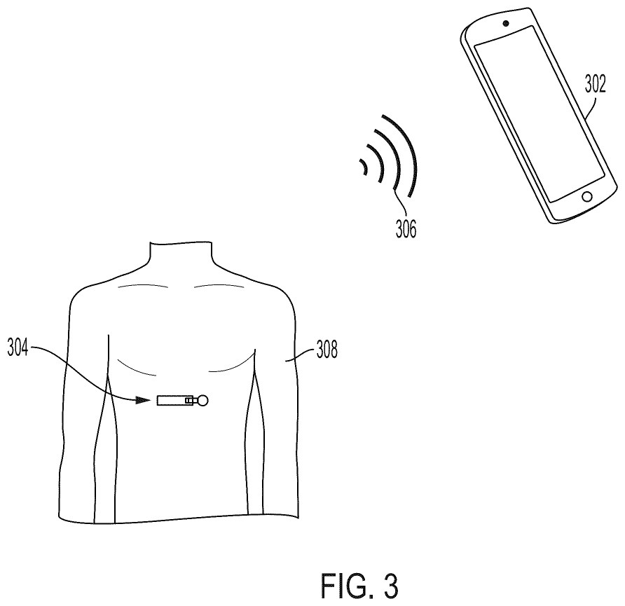

FIG. 3 is a system including a wearable sensor 304 configured to measure chest wall excursion in communication, such as via wireless signal 306, with processing device 302.

The wearable sensor may be positioned on the user 308 using an adhesive and/or strap, as described herein. The wearable sensor and processing device may be coupled using Bluetooth technology.

In some embodiments, processing of both the breathing signal and ventilatory thresholds can happen on a microprocessor physically connected to the sensor. The outputs of the processing can be streamed in real-time or saved and sent after the fact to another device.

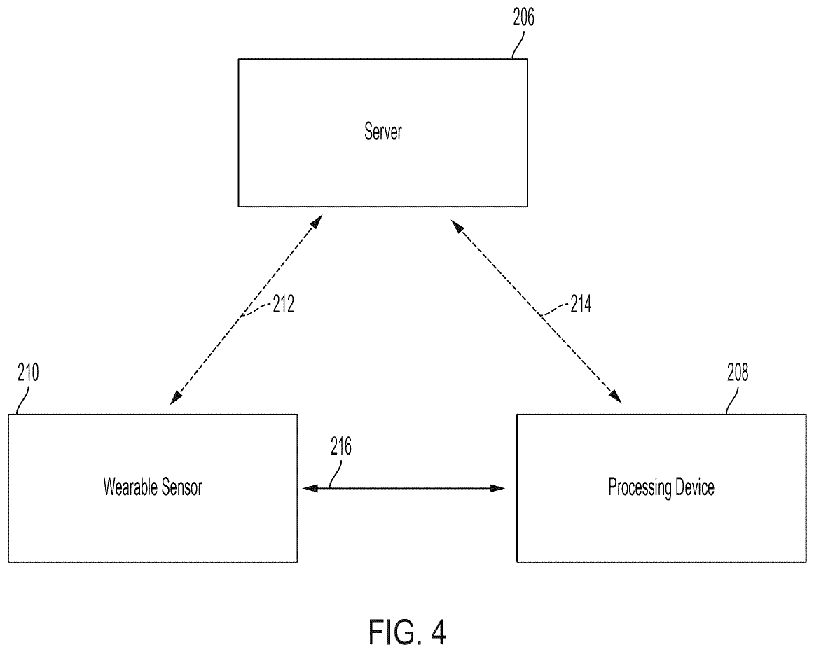

FIG. 4 is a block diagram of a system comprising a wearable sensor 210, a processing device 208, and optionally a server 206, for measuring ventilation thresholds and providing exercise recommendations. As described in relation to FIG. 3, the wearable sensor and processing device may be coupled via Bluetooth coupling. The wearable sensor and processing device may exchange data 212. The server may be a cloud server. The server and processing device may exchange data 214. The processing device and wearable sensor may exchange data 216.

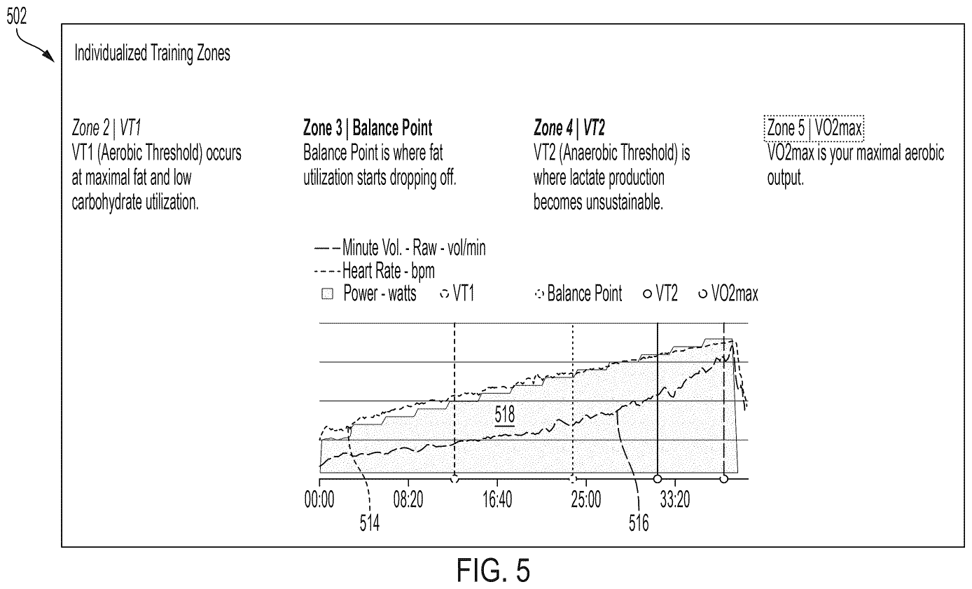

FIGS. 5-10 show fitness assessment and exercise recommendations to improve one or more of VT1, VT2, and VO2 max. While FIGS. 5-10 are shown with shading, when displayed to a user, a graphical user interface may include colors or other indicia to present information to the user.

FIG. 5 is a graphical user interface (GUI) 502 of a processing device illustrating presentation of ventilation threshold data. The GUI of the processing device may present information on Zone 2 and VT1, Zone 3 and a Balance Point, Zone 4 and VT2, and Zone 5 and VO2 max.

As shown in FIG. 5, the provided information may correspond to a graph presented on the GUI showing VT1, Balance Point, VT2, and VO2 max. The graph may plot power 518, heart rate 514, and/or minute ventilation 516 data. As apparent from the graph, FIG. 5 shows low, medium, and high ventilation thresholds. In this example, the naming convention of the low, medium, and high ventilation thresholds is VT1, Balance Point, and VT2 for purposes of providing an example.

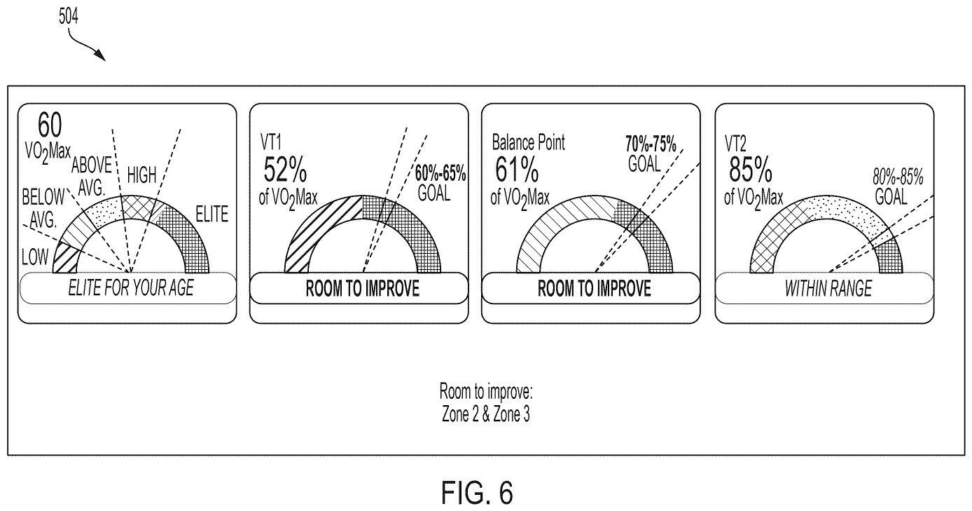

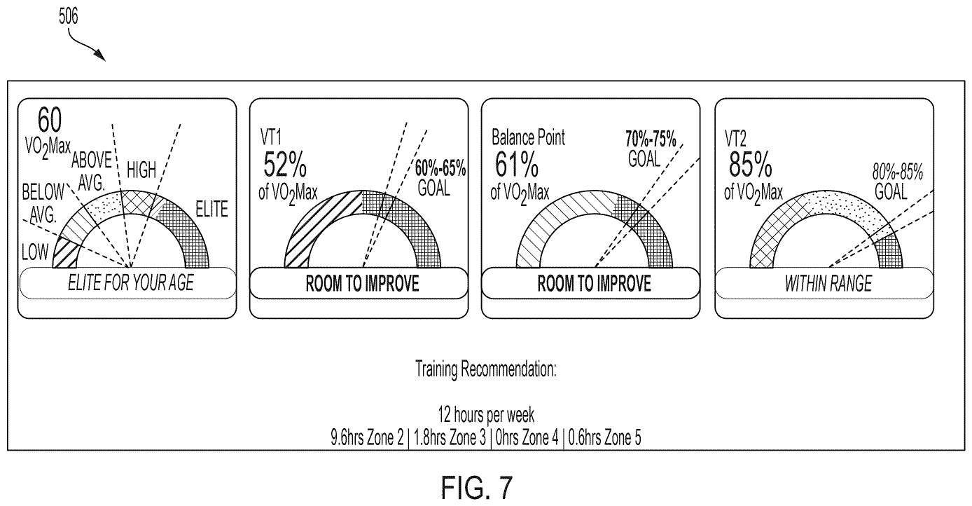

FIG. 6 is an example recommendation provided on a GUI 504 to a user for a training program. FIG. 6 shows a fitness assessment and corresponding exercise recommendation. As shown in FIG. 6, in addition to a recommendation provided to a user, the GUI may present VO2 max, VT1, Balance Point, and VT2 as well as pre-selected phrases based on one or more calculations described herein in relation to VO2 max, VT1, Balance Point, and VT2. The pre-selected phrases may include: “elite for your age,” “room to improve,” and “within range.” The recommendation may include the pre-selected phrase “room to improve” and an indication of one or more zones recommended for improvement. A portion of a ring may be associated with each of VO2 max, VT1, Balance Point, and VT2 and presented to a user with a portion of the ring filled in corresponding to a value indicating VO2 max, VT1, Balance Point, and VT2. As a non-limiting example, for a VT1 of 50% of VO2 max, the ring may be filled halfway. As shown in FIG. 6, a goal (or target) has been established for VT1, Balance Point, and VT2. The goal is represented by the region between the dashed lines in the graphical user interface elements of FIG. 6 corresponding to VT1, Balance Point, and VT2. This representation of the goal (or target) is used in other figures including FIGS. 7, 8, 9, and 10, as non-limiting examples.

FIG. 7 shows an example training recommendation provided on a GUI 506 to a user for a training program. FIG. 7 shows the fitness assessment described in relation to FIG. 6. FIG. 7 also shows presentation of an example training recommendation. The training recommendation may include a number of hours of recommended training per a period of time, such as a week. As shown, the example training recommendation includes a recommendation of 12 hours per week. The example training recommendation may also include a number of hours of recommended training in one or more zones. As shown, the example training recommendation includes a recommendation of 9.6 hours in Zone 2, 1.8 hours in Zone 3, 0.0 hours in Zone 4, and 0.6 hours in Zone 5.

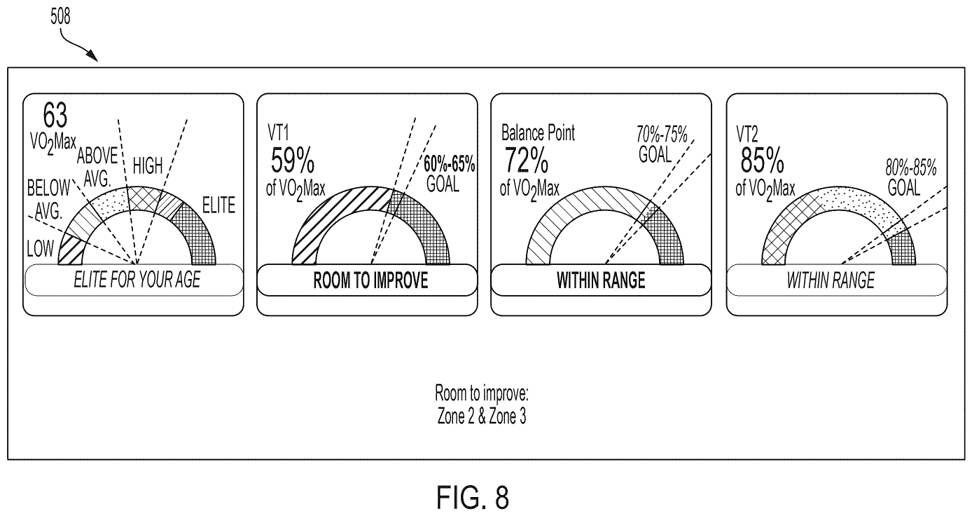

FIG. 8 shows an example recommendation provided on a GUI 508 to a user for a training program with improvements in each category relative to FIG. 6. FIG. 8 shows a fitness assessment and corresponding exercise recommendation of which Zones to improve.

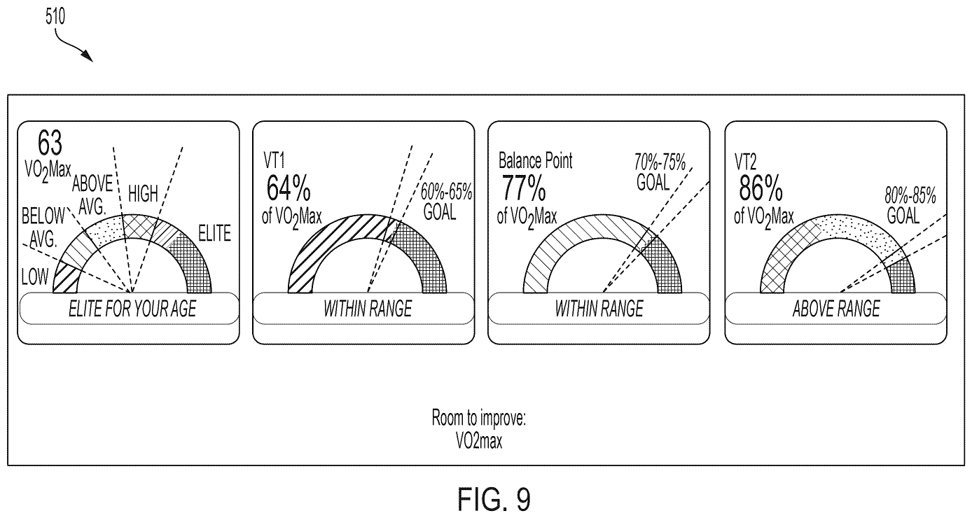

FIG. 9 shows an example recommendation provided on a GUI 510 to a user for a training program with improvements in some categories relative to FIG. 8. FIG. 9 shows a fitness assessment and corresponding exercise recommendation to improve VO2 max.

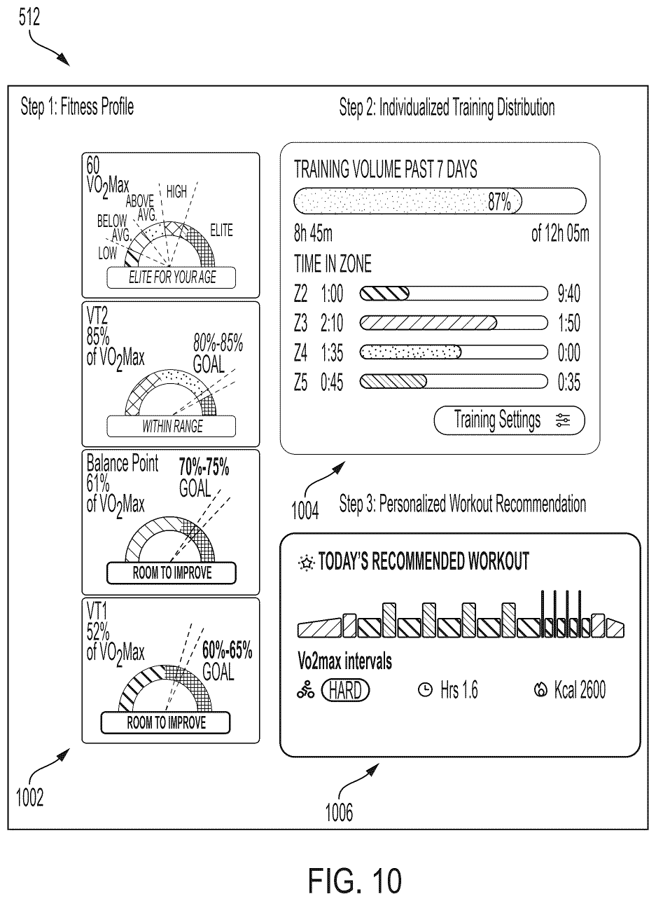

FIG. 10 shows a dashboard provided on a GUI 512 to a user including a fitness profile 1002, individualized training distribution 1004, and personalized workout recommendation 1006. The fitness profile 1002 may include one or more fitness assessments as described herein. The individualized training distribution 1004 may provide to a user a visualization of time spent in each zone over a period of time (e.g., in Zone 2, Zone 3, Zone 4, and Zone 5).

The individualized training distribution 1004 may include a training volume corresponding to a training recommendation previously provided, such as the training recommendation shown in FIG. 7. The training volume, for example, may show 8 h 45 m of a recommended weekly training of 12 h 05 m has been completed.

As shown in FIG. 10, the personalized workout recommendation 1006 may provide a workout recommendation corresponding to a present day. The duration, for example, may be 1.6 hours. The recommended workout information may include an indication or depiction of intervals for such a workout.

The fitness assessments and recommendations illustrated in FIGS. 5-10 may be made in various manners. For example, a person's VO2 max value may be incorporated which defines the total amount of daily workout energy expenditure (kilocalories) needed to maintain their current VO2max level. A formula may be used to generate a kilocalorie number.

Next, the distribution of time spent in each state may be defined. For example, the relative percentage of each threshold is compared to VO2 max.

What the inventors have observed is that diminishing returns occur when a given threshold reaches the following values:
Low ventilation threshold (such as VT1) ~65% VO2max
Medium ventilation threshold (such as Balance Point) ~75% VO2max
High ventilation threshold (such as VT2) ~85% VO2max

In some embodiments, if any threshold is below these reference levels, then it is a candidate to be emphasized in training to improve it. If all thresholds are within 1-2% of these reference levels, then increasing VO2 max may be emphasized.

Once the overall training recommendation is in place, the system can provide a workout recommendation, and in particular, can generate a daily workout recommendation that takes into account the current fitness level, status of the duration spent relative to each of their thresholds, which threshold bucket has the most amount of time to fill, and a number of other factors.

If a user is training more than one sport, for example running and cycling, the methodology of providing a recommendation may accommodate both sports within their overall recommendation based on their defined emphasis of each sport. Thresholds may be sport specific, so the recommendation can also account for the unique thresholds of each sport in making a recommendation for that specific sport. As a non-limiting example, an experienced runner will have running thresholds that translate to a certain ventilation, heart rate, pace/external load, and oxygen consumption at each threshold. This same person would have significantly different cycling thresholds in terms of the ventilation, heart rate, power/external load, and oxygen consumption at each threshold. Without wishing to be bound by any particular theory, this difference in thresholds can occur because the muscles of this person are trained and adapted for the demands of running, but this does not translate directly over to cycling. Therefore, if this person used their running thresholds as a benchmark in cycling workouts, this person would not be properly individualizing their training to that specific sport. Given this, the person may overtrain when using the same reference markers during a cycling workout. By accounting for the unique thresholds of each sport in making a recommendation for that specific sport, the system described herein may provide improved recommendations.

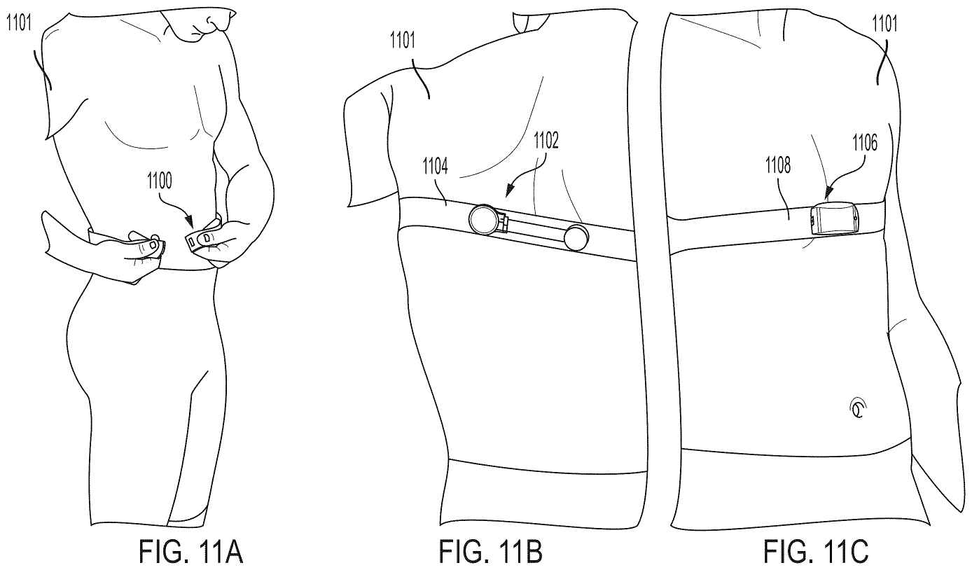

The wearable device, as described herein, may be implemented as shown in FIGS. 11-20 according to non-limiting embodiments of the present disclosure. The wearable device shown in and described in relation to FIGS. 11-20 may be used to obtain a chest excursion signal indicative of tidal volume.

FIGS. 11A-11C show positioning of a strap having one or more wearable sensors on a user 1101.

A strap, as described herein, may be implemented as strap 1100 as shown in FIG. 11A. A user 1101 may fasten the strap to achieve a snug fit for obtaining measurements as described herein.

Strap 1100 may include a stretchable portion and a non-stretchable portion. The stretchable portion 1104 may have a capacitive sensor 1102 coupled thereto as shown in FIG. 11B. The capacitive sensor may include a first and second end respectively coupled to the strap as shown. The capacitive sensor may operate to measure chest excursion as described previously herein. The wearable sensor includes a stretchable sensor, and the chest excursion signal is a varying signal representing stretching of the stretchable sensor as the user breathes. The stretchable portion may be correlated to tidal volume. As shown in FIG. 11B, the capacitive sensor may be positioned on a back side of a user 1101, although alternative positioning is possible. As shown in FIG. 11B, the capacitive sensor is facing an external surface of the strap. The strap may be configured to surround an abdomen or thorax of a user 1101, as shown in FIGS. 11A-11C. Strap 1100 may include a non-stretchable portion 1108 with a sensor 1106 coupled thereto as shown in FIG. 11C. The sensor 1106 may include a heart rate sensor as described herein. As shown in FIG. 11C, the heart rate sensor may be positioned on a front side of a user. In other embodiments, the heart rate sensor may be positioned on a back side of the user. The heart rate sensor may be configured to obtain heart rate data (such as in beats per minute). The ventilation signal, described elsewhere herein, may be obtained while the strap is positioned on the user 1101 to surround an abdomen or thorax of the user, such as shown in FIGS. 11B-11C.

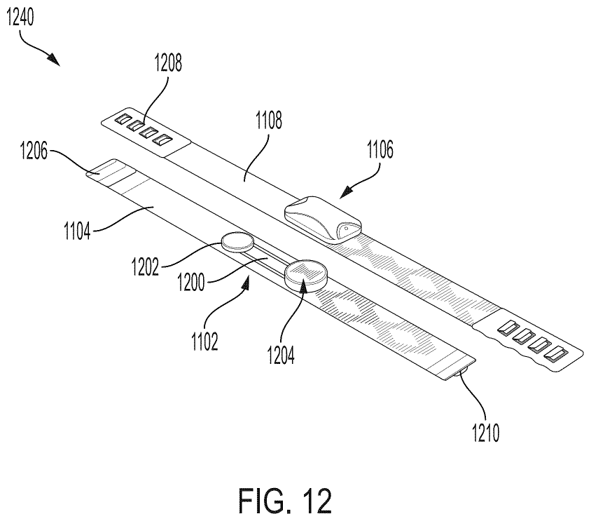

FIG. 12 shows a stretchable and non-stretchable portion of the wearable sensor 1240 of FIGS. 11A-11C.

As shown in FIG. 12, capacitive sensor 1102 may include a stretchable portion 1200 between a first end and second end thereof. The first and second end of the capacitive sensor may be coupled to the strap as shown. As described herein, the capacitive sensor may have a capacitance that changes based at least in part on a length of the stretchable portion of the capacitive sensor.

As also shown in FIG. 12, capacitive sensor 1102 may have its first end coupled to a module 1204. At the first end of capacitive sensor 1102, a first and second electrode may facilitate connection of the capacitive sensor to one or more electronic components of module 1204. Module 1204 may be configured to house such electronic components. The electronic components may include a battery, processing unit, control circuitry, inertial measurement units (IMUs), accelerometers, and Bluetooth connectivity elements.

The second end of the capacitive sensor may be coupled to a connector 1202. The second end of the capacitive sensor may have a hole formed therethrough. An adhesive may be applied to the second end where the hole is located for anchoring the second end of the capacitive sensor to the strap. The connector 1202 may secure the connection to the strap at the second end.

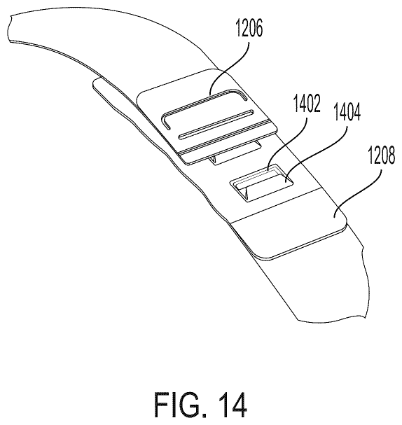

As shown in FIG. 12, the stretchable and non-stretchable portion of the wearable sensor may include one or more clip mechanisms. The clip mechanisms may couple the stretchable and non-stretchable portions to each other. In the non-limiting example of FIG. 12, the stretchable portion includes a clip mechanism 1206 at each end. The clip mechanisms 1206 may have protrusions 1210 extending therefrom. The protrusions may be configured to engage with a corresponding clip mechanism 1208 on another portion of the wearable sensor, such as on the non-stretchable portion as shown in FIG. 14.

As shown in FIG. 14, the clip mechanism 1208 of the non-stretchable portion may include a recessed region 1402. The recessed region 1402 may receive a protrusion of clip mechanism 1206. The clip mechanism 1208 may include retaining features 1404 to prevent disengagement of the clip mechanisms while positioned on a user. The retaining features may include one or more protrusions.

To achieve a snug fit, a user can engage the clip mechanisms using a recessed region of the one or more recessed regions of the non-stretchable portion. As shown in FIG. 14, when one recessed region is engaged, one or more other recessed regions may not be engaged. Referring to FIG. 12, while eight total recessed regions are shown for the wearable sensor 1240, fewer or more recessed regions may be included to provide additional or alternative sizing options.

When the wearable sensor has one or more fit adjustment portions as described herein, additional clip mechanisms can be used to attach such portions. Adding one or more fit adjustment portions can lengthen the non-stretchable portion since the additional adjustment portions may be non-stretchable segments.

The clip mechanisms for fit adjustment portions may be implemented using protrusions and corresponding recessed regions, such as those shown in FIG. 14. When a fit adjustment portion is added, a protrusion of the stretchable portion may engage with a recessed region of the fit adjustment portion at one end of the fit adjustment portion. At another end of the fit adjustment portion, a protrusion of the fit adjustment portion may engage with a recessed region of the non-stretchable portion.

As described herein, the clip mechanisms can provide a secure fit for a user when in use.

FIG. 13 shows an example of fit adjustment portions as may be used to adjust the fit of a strap having a stretchable respiration sensor. FIG. 13 shows adjustable no-stretch portions 1310 and non-adjustable stretch portion 1312.

The fit adjustment portions 1302 may be coupled to the stretchable and non-stretchable (no-stretch) portions as described herein.

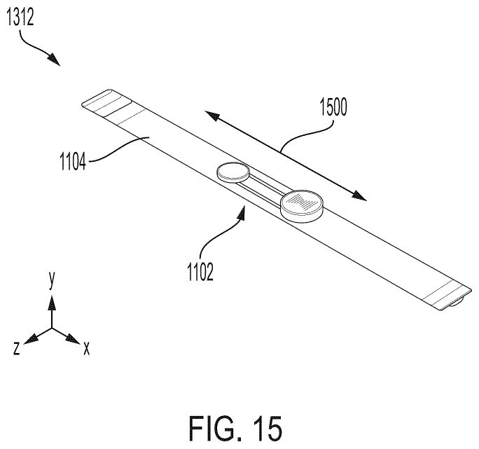

FIG. 15 shows a direction of stretchability for the stretchable portion or segment of the strap shown in FIGS. 11A-11C.

The direction of stretchability 1500 may be in a horizontal direction. The stretchable portion 1104 of the strap may stretch in the horizontal direction (e.g., X-direction).

The stretchable portion shown in FIG. 15 may have an initial fixed length that is not adjustable by a user. An amount of stretchable material for the stretchable segment of the strap may be a fixed amount. The stretchable portion may have a low residual elongation so that the length of the stretchable portion remains approximately the same over extended use. In some embodiments, the stretchable portion has a homogenous stretch modulus or elastic modulus, as described elsewhere herein. In some embodiments, the stretchable portion maintains a same stiffness over extended use. In some embodiments, the stretchable portion returns to a same initial length after tension is released, over extended use.

The stretchable portion may have an operating range between 0-100% of its initial length. The strap tension may be 8-12 Newtons (N) when positioned on a user, though other strap tension ranges are possible. At a tension above 12 N, the strap may not be positioned comfortably on a user. At a tension below 8 N, the strap may have a higher chance of falling down and having improper positioning for sensor measurements.

In some embodiments, the stretchable portion may be fabricated such that when the stretchable portion stretches between 10-100% of the initial length, the strap tension remains between 8 and 12 N and/or between 5 and 15 N. As a non-limiting example, if the stretchable portion is initially 10 inches, the stretchable portion may stretch 1 inch under such tension to enter an operating range, thereby avoiding falling off a user. On a force-elongation curve, such as shown in FIG. 16, the slope may be more gradual for such materials that can facilitate a secure fit without excess pressure on a user. In some embodiments, the strap exhibits a substantially constant force over a range of its stretched length. For example, over a range of 10%-100% of the straps stretched length, the strap may exhibit a substantially constant force (e.g., between 5N-15N). In some embodiments, the strap exhibits a substantially constant force over a range of its stretched length from 10%-75%, from 10%-50%, or 10%-30%. This property of constant force over a substantial range of stretch may facilitate the use of a single strap for people of multiple sizes without requiring mechanical adjustment.

The sensor coupled to the stretchable portion may also be stretched when the strap is positioned on a user. The sensor may stretch a same amount as the stretchable portion of the strap, as described herein. The sensor and stretchable portion of the strap may have a relative stretchability. The sensor may be sensitive to stretch and susceptible to damage after a particular stretch threshold. As a non-limiting example, the sensor may be configured to have a maximum stretch threshold of 120% to 130% of its initial length. To avoid passing such a threshold, the internal weave and/or material of the stretchable portion that the sensor may be coupled to can be selected to limit stretching thereof to 100%. In some embodiments, an externally applied limiter can prevent bypassing a maximum stretch threshold. In some embodiments, a Kevlar thread may restrict stretch when a stretchable portion reaches 100%. The stretchable portion may have a threshold of 100% stretch of its initial length. The Kevlar thread may have a total length that is twice the initial length of the stretchable portion. In cases where the Kevlar thread is fixed (e.g., via glue or stitching) in a snaked manner from one end to the other of the stretchable portion at rest, the Kevlar thread can restrict stretching beyond the threshold.

While the stretchable portion and sensor coupled thereto may undergo stretching up to 100%, for example, the non-stretchable portion may be configured to maintain approximately 0-100% stretch for a tension of 8-12 N.

The strap sizing described herein can facilitate maintaining a strap tension of 8-12 N among a variety of users whose chest and abdominal dimensions may vary.

FIG. 16 shows a force-elongation curve for a variety of materials. As shown and described herein, an ideal force-displacement range may be from a tension of 8 N to 12 N. The relationship for example materials 1 to 7 is shown in FIG. 16. As shown, #1 material 1602, #2 material 1604, #3 material 1608, #4 material 1610, #5 material 1606, #6 material 1612, and #7 material 1614 have different force-elongation curves.

FIG. 17 illustrates a diagram representing the capacitive sensor in a relaxed and stretched state. The capacitive sensor may be stretched in the direction of stretchability 1500 as shown. The capacitive sensor may have a capacitance that changes based at least in part on a length of the stretchable portion. The one or more changes in capacitance of the capacitive sensor, as described herein, may be based on the stretchable portion of the capacitive sensor being stretched by 5%-40%, 20%-40%, 5%-60%, or any range or value therein. In some embodiments, the stretchable portion of the capacitive sensor may be stretched by 10%-70%, or any range or value therein.

When the capacitive sensor is relaxed, the capacitance can be calculated as C₀. Using the following Equation 1, it can be found that the initial capacitance is less than the capacitance in a stretched state. In Equation 1, C₀ represents the initial capacitance, co represents the vacuum permittivity, εᵣ represents the relative permittivity of the dielectric, A₀ represents the initial cross-sectional area, d₀ represents an initial distance of separation, C represents the capacitance in a stretched state, A represents the cross-sectional area in a stretched state, and d represents a distance of separation in a stretched state.

$${C}_{0} = {ε}_{0} {ε}_{r} \frac{{A}_{0}}{{d}_{0}} < C = {ε}_{0} {ε}_{r} \frac{A}{d} \qquad ( Equation 1 )$$

FIG. 18 shows a cross-sectional view of layers of the capacitive sensor. The silicone (or other material as described herein) may have conductive particles (e.g., carbon black) on each side to create two separated conductive surfaces. FIG. 18 shows a relaxed distance 1802 and a stretched distance 1804 with separation of layers 1806 and 1808.

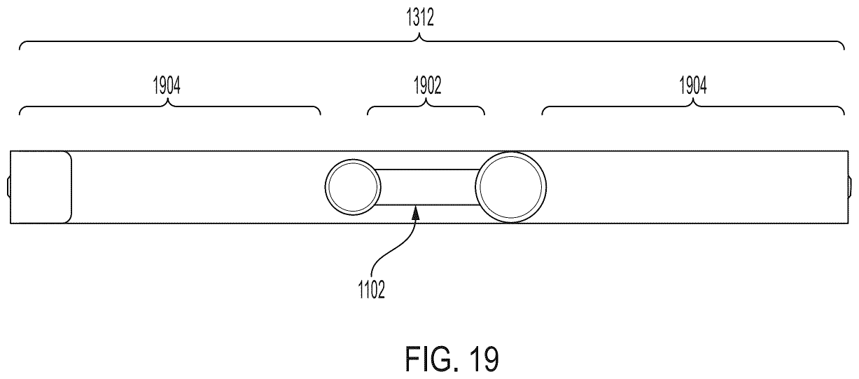

FIG. 19 shows different portions of the stretchable portion of the strap. As a non-limiting example, the stretchable portion may have one sensing portion 1902 and two non-sensing portions 1904. The stretchable portion, such as non-adjustable stretch portion 1312, may have a non-adjustable length as described herein. The sensing portion length can be equal to or less than the non-adjustable stretch portion. In some embodiments, non-sensing portions 1904 may not be stretchable. In such an embodiment, non-adjustable stretch portion 1312 may include only sensing portion 1902 but not non-sensing portions 1904. By having non-sensing portions 1904 be non-stretchable portions, the inventors recognized that when an impediment impinges on the non-sensing portions, having them be a non-stretchable material can improve measurements in some instances. When both non-sensing portions are stretchable such as shown in FIG. 19 and an impediment impinges on the non-sensing portions, the system can account for the impediment with calibration.

As shown in FIG. 19, the stretchable portion of the capacitive sensor may have a rectangular shape. The capacitive sensor may be a straight segment. In some embodiments, the stretchable portion of the capacitive sensor may be circular, oval, or triangular.

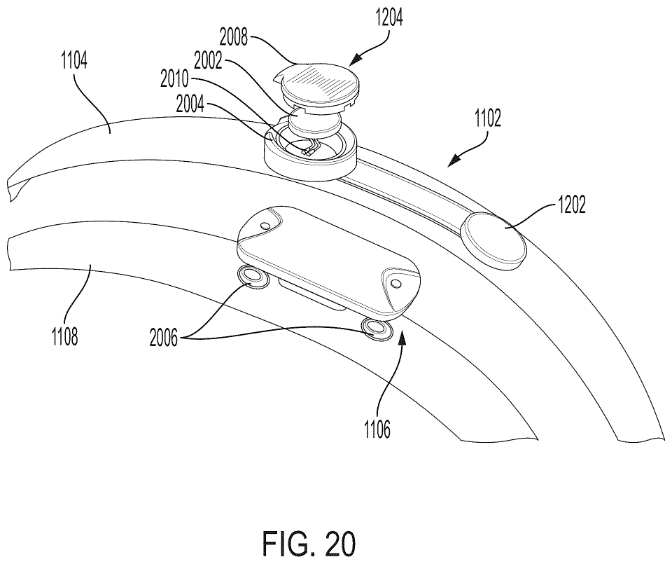

FIG. 20 shows an exploded view of the respective sensors of the stretchable and non-stretchable portions of FIGS. 11A-11C.

As shown in FIG. 20, sensor 1106 may be removably coupled to the non-stretchable portion 1108 of the strap. The strap and sensor may be coupled using one or more fasteners 2006. The fasteners may include a button such that the sensor can be removed from the strap. In some embodiments, the fasteners may include Velcro, snaps, or temporary adhesive. The fasteners may provide for permanent attachment such that the sensor cannot be removed from the strap by a user.

As shown in FIG. 20, module 1204 may include a housing 2004 configured to house electronic components, such as a battery. The housing may include a lid 2008. A battery 2002 may be disposed within the housing 2004 as shown in the exploded view. The battery may be a coin battery or a permanently fixed Lithium Polymer (LiPo) battery.

As described herein, a first end of the capacitive sensor may be coupled to the module having the housing 2004. Conductive elements 2010 may be disposed within the housing to facilitate electrical connection between the capacitive sensor and the battery, as a non-limiting example.

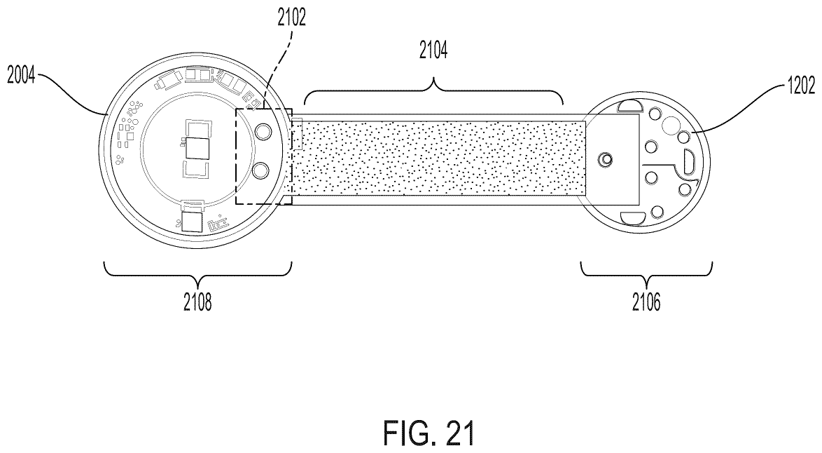

FIG. 21 shows inner sides of a module and connector coupled to the capacitive sensor. FIG. 21 shows electrodes 2102 that couple the capacitive sensor to the one or more electronic components in the housing 2004. As shown in FIG. 21, the capacitive sensor may include a capacitive sensor dielectric 2104. The capacitive sensor dielectric may include silicone, urethane or rubber, as described herein. The capacitive sensor dielectric may be coated on two surfaces, as a non-limiting example, with a conductive material or conductive particles.

The capacitive sensor may include two mechanical anchor points 2106 and 2108 as shown in FIG. 21. As described in relation to FIG. 12, an end of the capacitive sensor may have a hole formed therethrough. The connector 1202 may secure the connection to the strap at that end.

Thus, the stretchable wearable sensors described herein may be beneficially configured to provide an accurate and reliable ventilation signal based on chest wall excursions. The sensors may provide consistent data over the course of an exercise session and day-to-day due to their characteristics of consistent stretch. The lightweight nature of the sensors also makes it practical to wear them in day-to-day activities and for long periods of time, facilitating the collection of meaningful data which can be drawn upon to provide recommendations for improving the user's fitness.

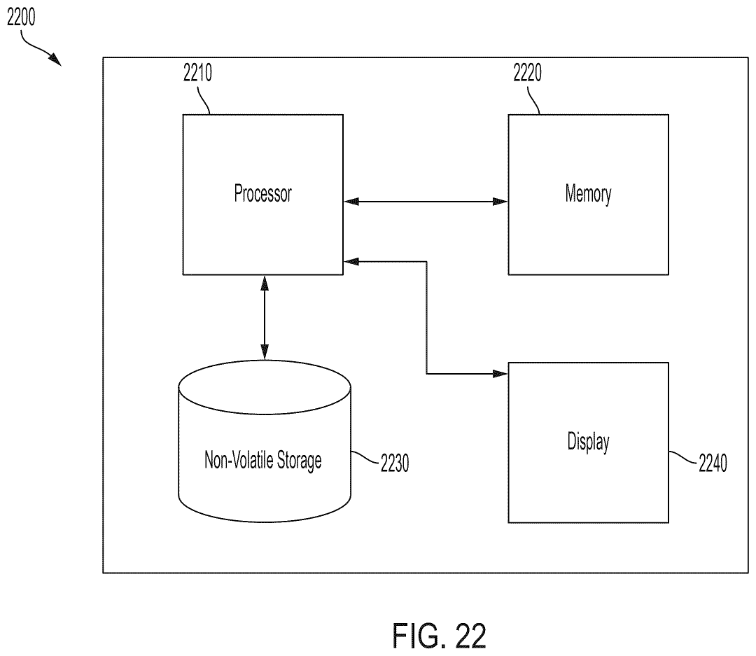

As should be appreciated from the description above, various aspects of the invention may be implemented on one or more computer systems, such as the exemplary system 2200 shown in FIG. 22. Computer system 2200 includes processor 2210, memory 2220, non-volatile storage 2230, and a display 2240. The processor may be a processing device of the types described herein. The processor may perform one or more functions of the processing devices or wearable sensor electronics described herein. Instructions for the processor may be stored, for example, in non-volatile storage 2230, and data may be stored, for example, in memory 2220. Alternatively, instructions for the processor may be stored in the memory 2220 and data may be stored in the non-volatile storage 2230. In still further embodiments, the memory 2220 and non-volatile storage 2230 may both store instructions and/or data. The memory and/or non-volatile storage may be a disk (e.g., an optical disk), a compact disk (cd), a solid-stage memory, or any other memory, and in some embodiments may be configured to store process-executable instructions which, when executed by the processor 2210 cause the processor to perform any of the methods described herein. It should be appreciated that the memory 2220 and non-volatile storage 2230 are non-transitory media in at least some embodiments. Optionally, a display 2240 may be provided, for example so that personnel may interact with the processor and view data output by the processor.

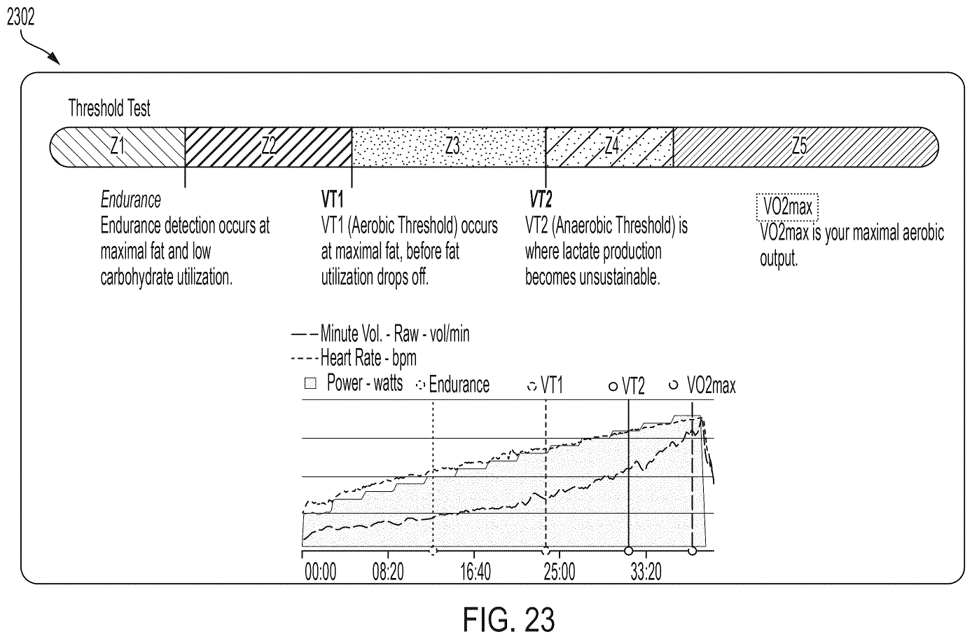

As described herein, ventilation thresholds such as low, medium, and high as described above, or referred to by names such as VT1, BP, or VT2, and VO2 max may be identified in some embodiments. In alternative embodiments, the naming conventions of the thresholds may be different. FIGS. 23-28 show example displays provided on a GUI to a user in which Endurance, VT1, VT2, and VO2 max are identified. Compared to FIG. 5, as a non-limiting example, FIG. 23 shows an alternative naming convention using “Endurance” where “VT1” was identified in FIG. 5 and with “VT1” in FIG. 23 where “Balance Point” was identified in FIG. 5. In each example, the corresponding inflection point is detected at a same point, but merely the name of the threshold may be different. The corresponding inflection points may correspond to low, medium, and high ventilation thresholds. Therefore, it should be appreciated that while different naming conventions may be used, the identified points of a measured signal that may reference physiological markers are the same. Moreover, while examples of Graphical User Interfaces with “Balance Point” and “Endurance” are provided in the figures, as described herein, physiological parameters including physiological thresholds may be referred to using other terms. Corresponding graphical displays may be generated and provided to a user with such alternative naming conventions to capture the thresholds described herein.

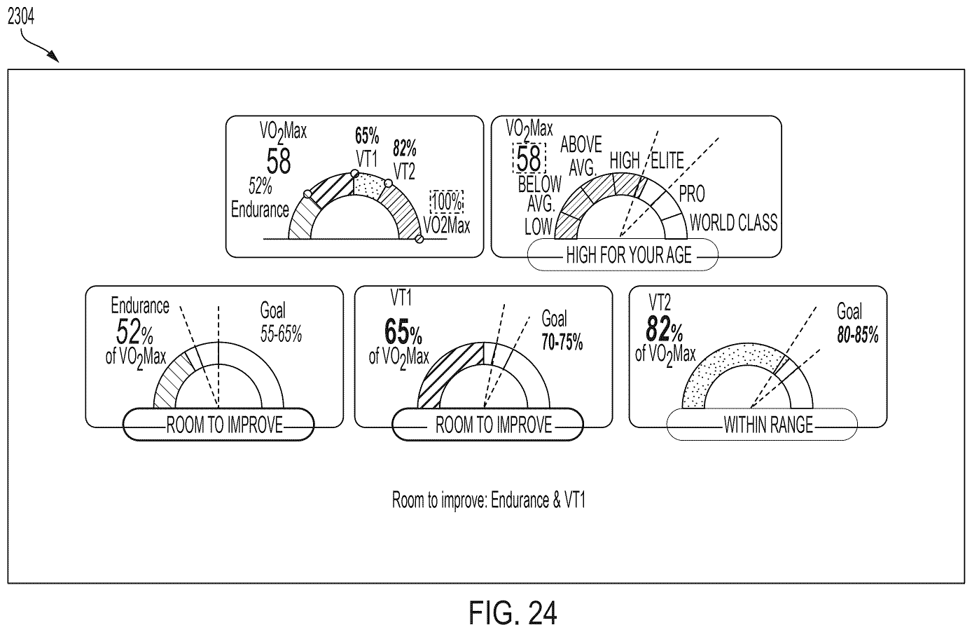

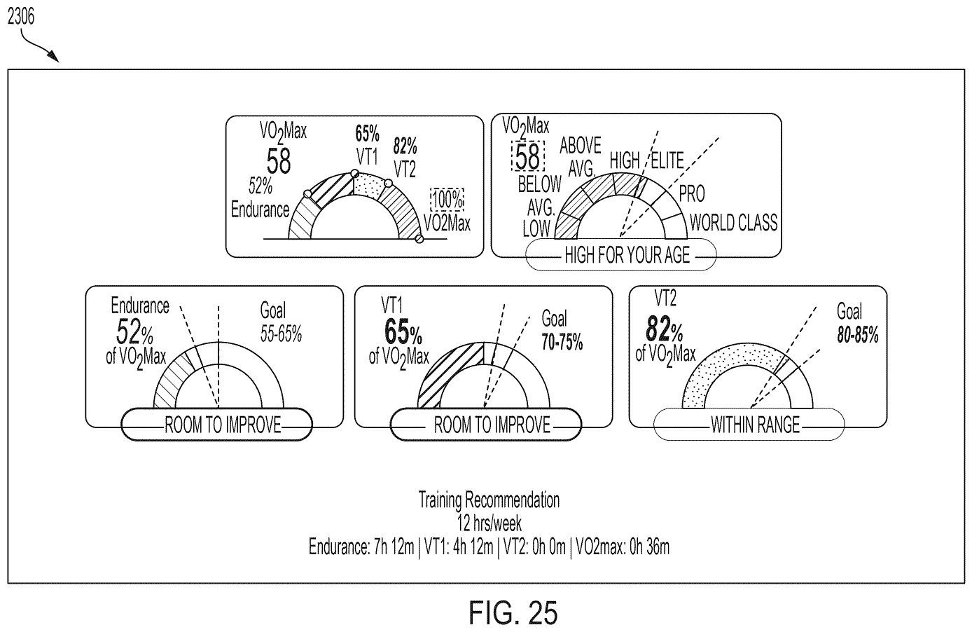

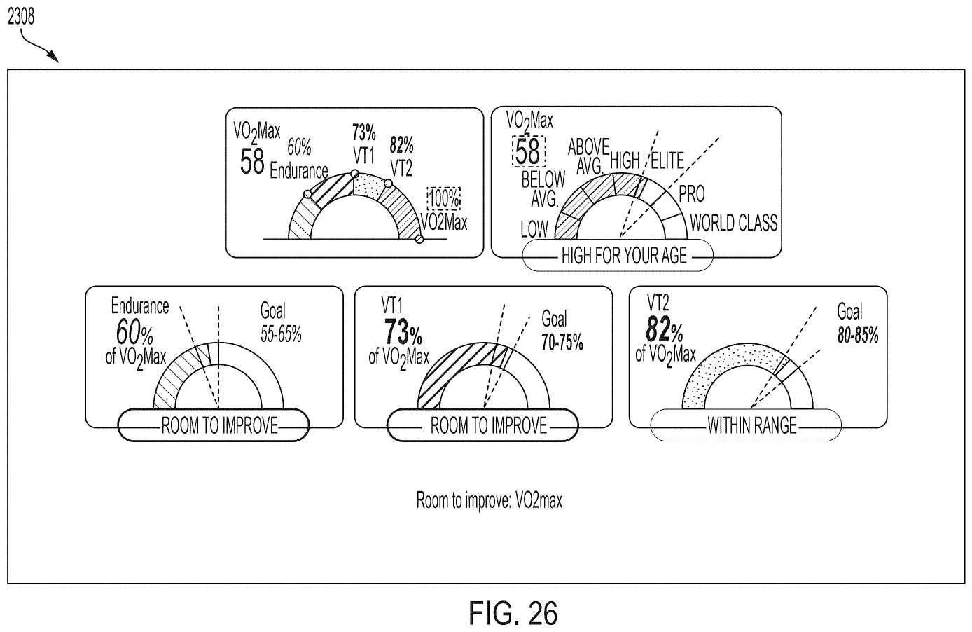

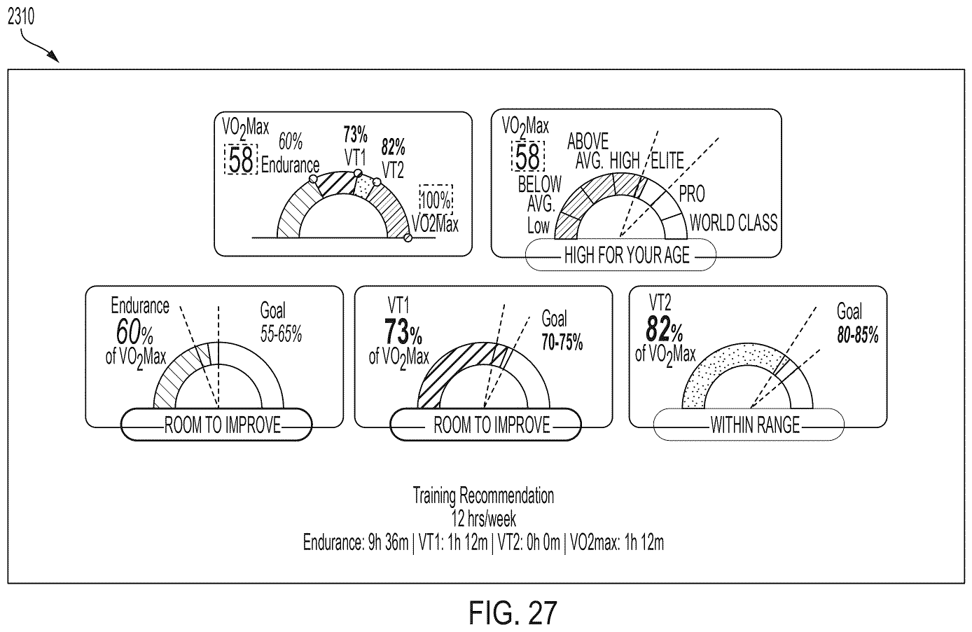

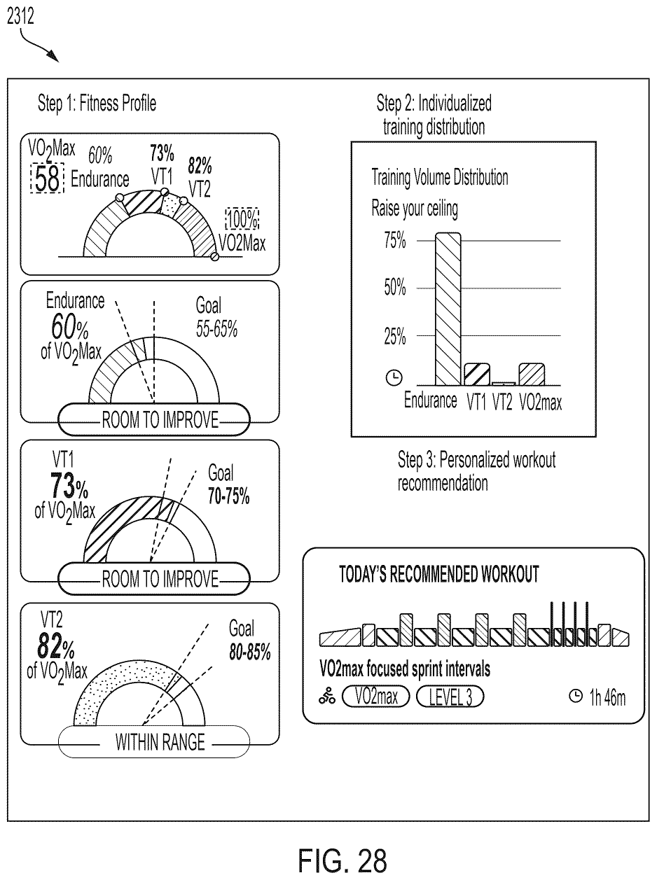

FIG. 23 is a graphical user interface (GUI) 2302 of a processing device illustrating presentation of ventilation threshold data. FIG. 24 is an example recommendation provided on a GUI 2304 to a user for a training program. FIG. 25 shows an example training recommendation provided on a GUI 2306 to a user for a training program. FIG. 26 shows an example recommendation provided on a GUI 2308 to a user for a training program with improvements in each category. FIG. 27 shows an example recommendation provided on a GUI 2310 to a user for a training program. FIG. 28 shows a dashboard provided on a GUI 2312 to a user including a fitness profile, individualized training distribution, and personalized workout recommendation.

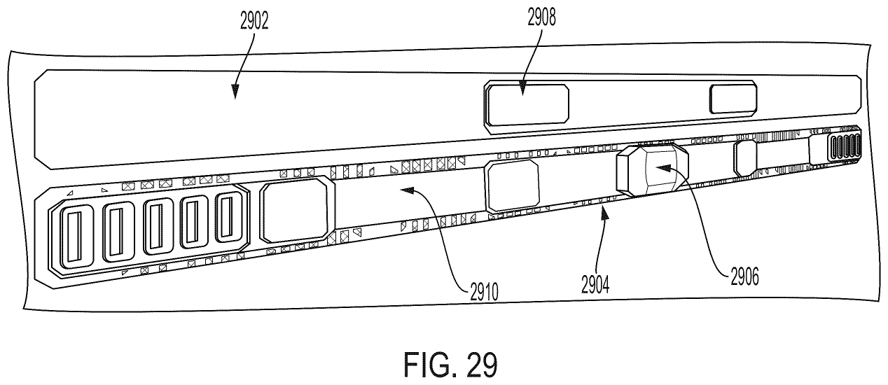

While some examples of the system described herein (including a wearable sensor) include multiple housings or modules, such as shown in FIG. 12, the system is not so limited. FIG. 29 shows an example system including a wearable sensor and a single housing 2906 for electronics, according to some embodiments. The single housing 2906 may house a heart rate measuring unit, a battery, processing unit, control circuitry, inertial measurement units (IMUs), accelerometers, and Bluetooth connectivity elements. One or more measurements from an inertial measurement unit, an accelerometer, and/or a heart rate sensor coupled to the strap may be obtained.

As shown in FIG. 29, first and second straps 2902 and 2904 may be provided that can be coupled together. As described in relation to FIG. 13, additional fit adjustment portions (not shown in FIG. 29) may be included.

The first strap 2902 may include one or more electrodes 2908 for heart rate measurements. Second strap 2904 may include capacitive sensors 2910. Capacitive sensors 2910 may have the same material and functionality as capacitive sensor 1102 described elsewhere herein. The capacitive sensors 2910 may be integrated on stretchable portions of the strap. In some embodiments, the stretchable portions may range from 5 cm to 10 cm each.

As shown in FIG. 29, two capacitive sensors 2910 may be provided on opposite sides of the housing 2906. The inventors appreciated that having two capacitive sensors provides redundancy such that if one sensor is not providing accurate readings, the other of the two capacitive sensors may be used. As an example, one of the two capacitive sensors may be prevented from stretching due to an external force, such as from a user laying on their side or sitting on a bike with a seat contacting a portion of the strap, and therefore may not provide useful readings. Further, if both sensors are providing sensor data, the sensor output may be more accurate with two sets of measurements as opposed to one set of measurements. The signals corresponding to the two or more sets of measurements may be combined with averaging or another weighting scheme and/or by summing the two signals to merge them into one signal.

Aspects of the technology may be implemented by a computer readable storage medium comprising computer readable instructions configured to be executed by a processor to cause the processor to perform any of the methods described herein.

Aspects of the technology described herein may provide various benefits. Below are some examples. Not all embodiments necessarily provide all listed benefits and benefits other than those may be realized in at least some embodiments.

For example, aspects of the present technology provide accurate and consistent measurements of ventilation behavior of a user in a convenient and comfortable wearable device. The accurate and consistent measurements allow for accurate measurement of physiological changes in the user during exertion. Cardiometabolic performance may be improved with recommendations specific to the user's physiological changes experienced during exertion. Because the recommendations may be tailored to the user's unique ventilation performance, improvement in the user's cardiometabolic performance may be made more likely and may occur at a faster rate than compared to recommendations provided based on calculated ventilation thresholds or recommendations based on population statistics.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

[symbol]

1. A wearable device, comprising:
a strap; and
a capacitive sensor coupled to the strap, wherein the capacitive sensor comprises:
a first end coupled to the strap;
a second end coupled to the strap; and
a stretchable portion between the first end and the second end,
wherein the capacitive sensor has a capacitance that changes based at least in part on a length of the stretchable portion.

2. The wearable device of claim 1, wherein the wearable device is configured to measure chest wall excursion based at least in part on a change in the capacitance of the capacitive sensor.

3. The wearable device of claim 1, wherein the strap comprises a stretchable segment, and wherein an amount of stretchable material for the stretchable segment of the strap is a fixed amount.

4. The wearable device of claim 1, wherein the first end of the capacitive sensor is coupled to a module having a housing.

5. The wearable device of claim 4, further comprising an inertial measurement unit and/or an accelerometer in the housing of the module.

6. The wearable device of claim 1, further comprising a heart rate sensor coupled to the strap.

7. The wearable device of claim 1, wherein the capacitive sensor is facing an external surface of the strap.

8. The wearable device of claim 1, wherein the capacitive sensor comprises silicone, urethane or rubber.

9. The wearable device of claim 1, wherein the strap is configured to surround an abdomen or thorax of a user.

10-11. (canceled)

12. A method for measuring chest wall excursion using a wearable device comprising a capacitive sensor coupled to a strap, the method comprising:
obtaining a ventilation signal using the wearable device, wherein the ventilation signal is based at least in part on one or more changes in capacitance of the capacitive sensor;
using one or more points of the ventilation signal to measure the chest wall excursion; and
obtaining tidal volume based on the chest wall excursion.

13. The method of claim 12, wherein the one or more points of the ventilation signal are inflection points.

14. The method of claim 12, wherein the capacitive sensor comprises a stretchable portion correlated to the tidal volume.

15. (canceled)

16. The method of claim 12, further comprising measuring one or more ventilatory thresholds based in part on the tidal volume.

17. The method of claim 16, wherein the one or more ventilatory thresholds include VT1 and VT2.

18. The method of claim 12, wherein the ventilation signal is obtained while the strap is positioned on a user to surround an abdomen or thorax of the user.

19. The method of claim 12, further comprising obtaining one or more measurements from an inertial measurement unit, an accelerometer, and/or a heart rate sensor coupled to the strap.

20. The method of claim 12, further comprising filtering the ventilation signal to obtain the one or more points.

21. The method of claim 12, further comprising, using a trained model to extract one or more thresholds from a minute ventilation signal.

22. The method of claim 16, wherein measuring one or more ventilatory thresholds based on the tidal volume comprises measuring the one or more ventilatory thresholds based on a minute ventilation signal.

23. (canceled)

24. The method of claim 12, further comprising, using linear regression, two-line best fit, three-line best fit, four-line best fit, or a Gaussian model that assess break point to extract one or more thresholds of a minute ventilation signal.

25. The method of claim 14, wherein the one or more changes in capacitance of the capacitive sensor are based on the stretchable portion of the capacitive sensor being stretched by 5%-40%.

26-40. (canceled)
